SEQUENCE LISTING (94 sequences)

SEQ ID NO: 1 - protein, 358 aa, Pseudomonas fluorescens
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEGNFKALFESIERDQVRRGVLTAD

SEQ ID NO: 2 - protein, 358 aa, Artificial Sequence
  HPPD mutant - PfG336W
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEWNFKALFESIERDQVRRGVLTAD

SEQ ID NO: 3 - protein, 358 aa, Artificial Sequence
  HPPD mutant - PfHPPDEvo37
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREWRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEWNFKALFESIERDQVRRGVLTAD

SEQ ID NO: 4 - protein, 358 aa, Artificial Sequence
  HPPD mutant - C0234E6
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSWNFKALFESIERDQVRRGVLTAD

SEQ ID NO: 5 - protein, 358 aa, Artificial Sequence
  HPPD mutant - C024H11
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRILLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEWNFKALFESIERDQVRRGVLTAD

SEQ ID NO: 6 - protein, 358 aa, Artificial Sequence
  HPPD mutant - PfHPPDEvo33
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFKALFESIERDQVRRGVLTAD

SEQ ID NO: 7 - protein, 358 aa, Artificial Sequence
  HPPD mutant - PfHPPDEvo36
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFTQLFESIERDQVRRGVLTAD

SEQ ID NO: 8 - protein, 358 aa, Artificial Sequence
  HPPD mutant - PfHPPDEvo40
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFKELFESIERDQVRRGVLTAD

SEQ ID NO: 9 - protein, 358 aa, Artificial Sequence
  HPPD mutant - CO210d10
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFTALFESIERDQVRRGVLTAD

SEQ ID NO: 10 - protein, 358 aa, Artificial Sequence
  HPPD mutant - CO212f3
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGGWNFKVLFESIERDQVRRGVLTAD

SEQ ID NO: 11 - protein, 358 aa, Artificial Sequence
  HPPD mutant - C644
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGIWNFKALFESIERDQVRRGVLTAD

SEQ ID NO: 12 - protein, 358 aa, Artificial Sequence
  HPPD mutant - C645
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGIGNFKALFESIERDQVRRGVLTAD

SEQ ID NO: 13 - protein, 358 aa, Artificial Sequence
  HPPD mutant - c0218A5
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFAALFESIERDQVRRGVLTAD

SEQ ID NO: 14 - protein, 358 aa, Artificial Sequence
  HPPD mutant - C0216C6
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFTALFESIERDQVRRGVLTAD

SEQ ID NO: 15 - protein, 358 aa, Artificial Sequence
  HPPD mutant - c0213H10
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGHWNFTQLFESIERDQVRRGVLTAD

SEQ ID NO: 16 - protein, 358 aa, Artificial Sequence
  HPPD mutant - PfHPPDEvo41
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFAQLFESIERDQVRRGVLTAD

SEQ ID NO: 17 - protein, 358 aa, Artificial Sequence
  HPPD mutant - C0228G9
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARIFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEWNFKALFESIERDQVRRGVLTAD

SEQ ID NO: 18 - protein, 358 aa, Artificial Sequence
  HPPD mutant - C0232D2
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREWSYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEWNFKALFESIERDQVRRGVLTAD

SEQ ID NO: 19 - protein, 358 aa, Artificial Sequence
  HPPD mutant - C0234A4
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGAWNFSQLFESIERDQVRRGVLTAD

SEQ ID NO: 20 - protein, 358 aa, Artificial Sequence
  HPPD mutant - C0235F6
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGASNFSQLFESIERDQVRRGVLTAD

SEQ ID NO: 21 - protein, 358 aa, Artificial Sequence
  HPPD mutant - C0235E2
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFSQLFESIERDQVRRGVLTAD

SEQ ID NO: 22 - protein, 358 aa, Artificial Sequence
  HPPD mutant - C0236H7
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSWNFKELFESIERDQVRRGVLTAD

SEQ ID NO: 23 - protein, 358 aa, Artificial Sequence
  HPPD mutant - C0236F8
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGASNFTQLFESIERDQVRRGVLTAD

SEQ ID NO: 24 - protein, 358 aa, Artificial Sequence
  HPPD mutant - C0240D2
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGAWNFKALFESIERDQVRRGVLTAD

SEQ ID NO: 25 - protein, 358 aa, Artificial Sequence
  HPPD mutant - C0240D12
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFTELFESIERDQVRRGVLTAD

SEQ ID NO: 26 - protein, 358 aa, Artificial Sequence
  HPPD mutant - C0242D4
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFKQLFESIERDQVRRGVLTAD

SEQ ID NO: 27 - protein, 358 aa, Artificial Sequence
  HPPD mutant - C0244A2
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFKALFESIERDQVRRGVLTAD

SEQ ID NO: 28 - protein, 358 aa, Artificial Sequence
  HPPD mutant - C0244F5
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFTALFESIERDQVRRGVLTAD

SEQ ID NO: 29 - protein, 358 aa, Artificial Sequence
  HPPD mutant - C0247B6
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGQSNFKELFESIERDQVRRGVLTAD

SEQ ID NO: 30 - protein, 358 aa, Artificial Sequence
  HPPD mutant - C0247H7
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREWSYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFTELFESIERDQVRRGVLTAD

SEQ ID NO: 31 - protein, 358 aa, Artificial Sequence
  HPPD mutant - C0252F11
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFTQLFESIERDQVRRGVLTAD

SEQ ID NO: 32 - protein, 358 aa, Artificial Sequence
  HPPD mutant - C0255B12
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREWCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFKQLFESIERDQVRRGVLTAD

SEQ ID NO: 33 - protein, 358 aa, Artificial Sequence
  HPPD mutant - C0255C1
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFTELFESIERDQVRRGVLTAD

SEQ ID NO: 34 - protein, 357 aa, Artificial Sequence
  HPPD mutant - C0255C3
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFELFESIERDQVRRGVLTAD

SEQ ID NO: 35 - protein, 358 aa, Artificial Sequence
  HPPD mutant - C0255E6
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESSYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFTELFESIERDQVRRGVLTAD

SEQ ID NO: 36 - protein, 358 aa, Artificial Sequence
  HPPD mutant - C0255E10
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFTALFESIERDQVRRGVLTAD

SEQ ID NO: 37 - protein, 358 aa, Artificial Sequence
  HPPD mutant - C0256B1
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGESNFTELFESIERDQVRRGVLTAD

SEQ ID NO: 38 - protein, 358 aa, Artificial Sequence
  HPPD mutant - C0256G11
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESSYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFKQLFESIERDQVRRGVLTAD

SEQ ID NO: 39 - protein, 358 aa, Artificial Sequence
  HPPD mutant - C0256H4
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGCYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFSELFESIERDQVRRGVLTAD

SEQ ID NO: 40 - protein, 358 aa, Artificial Sequence
  HPPD mutant - C0257C5
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFKQLFESIERDQVRRGVLTAD

SEQ ID NO: 41 - protein, 358 aa, Artificial Sequence
  HPPD mutant - C0260E11
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFTELFESIERDQVRRGVLTAD

SEQ ID NO: 42 - protein, 358 aa, Artificial Sequence
  HPPD mutant - C0260C6
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESSYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFTELFESIERDQVRRGVLTAD

SEQ ID NO: 43 - protein, 358 aa, Artificial Sequence
  HPPD mutant - C0262C4
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEWNFKQLFESIERDQVRRGVLTAD

SEQ ID NO: 44 - protein, 358 aa, Artificial Sequence
  HPPD mutant - C0262F11
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGSSNFKQLFESIERDQVRRGVLTAD

SEQ ID NO: 45 - protein, 358 aa, Artificial Sequence
  HPPD mutant - C0263B7
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREACYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFTQLFESIERDQVRRGVLTAD

SEQ ID NO: 46 - protein, 358 aa, Artificial Sequence
  HPPD mutant - C0263G12
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGQWNFSELFESIERDQVRRGVLTAD

SEQ ID NO: 47 - protein, 358 aa, Artificial Sequence
  HPPD mutant - C0261H2
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFSALFESIERDQVRRGVLTAD

SEQ ID NO: 48 - protein, 358 aa, Artificial Sequence
  HPPD mutant - C0264G5
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGQSNFTELFESIERDQVRRGVLTAD

SEQ ID NO: 49 - protein, 358 aa, Artificial Sequence
  HPPD mutant - C0264G7
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFRESRYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFKQLFESIERDQVRRGVLTAD

SEQ ID NO: 50 - protein, 358 aa, Artificial Sequence
  HPPD mutant - C0266A11
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREGSYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPSNFTQLFESIERDQVRRGVLTAD

SEQ ID NO: 51 - protein, 357 aa, Artificial Sequence
  HPPD mutant - Axmi305H-Evo40
MNAVAKIEQHNPIGTDGFEFVEFTAPDAKGIEQLRQLFNMMGFTETAKHRSKEVFLFQQNDINIVLNGSPTGHVHEFALKHGPSACAMAFRVKNASQAAAYAESQGAKLVGSHANFGELNIPSLEGIGGSLLYLVDRYGDRSIYDVDFEFIEGRSANDNSVGLTYIDHLTHNVKRGQMDVWSGFYERIANFREIRYFDIEGKLTGLFSRAMTAPCGKIRIPINESADDTSQIEEFIREYHGEGIQHIALTTDDIYATVRKLRDNGVKFMSTPDTYYEKVDTRVAGHGEPLEQLRELNLLIDGAPGDDGILLQIFTDTVIGPIFFEIIQRKGNQGFGPSNFKELFESIEEDQIRRGVI

SEQ ID NO: 52 - protein, 357 aa, Artificial Sequence
  HPPD mutant - Axmi305H-Evo41
MNAVAKIEQHNPIGTDGFEFVEFTAPDAKGIEQLRQLFNMMGFTETAKHRSKEVFLFQQNDINIVLNGSPTGHVHEFALKHGPSACAMAFRVKNASQAAAYAESQGAKLVGSHANFGELNIPSLEGIGGSLLYLVDRYGDRSIYDVDFEFIEGRSANDNSVGLTYIDHLTHNVKRGQMDVWSGFYERIANFREIRYFDIEGKLTGLFSRAMTAPCGKIRIPINESADDTSQIEEFIREYHGEGIQHIALTTDDIYATVRKLRDNGVKFMSTPDTYYEKVDTRVAGHGEPLEQLRELNLLIDGAPGDDGILLQIFTDTVIGPIFFEIIQRKGNQGFGPWNFAQLFESIEEDQIRRGVI

SEQ ID NO: 53 - protein, 358 aa, Artificial Sequence
  HPPD mutant - Axmi305H-Evo40
MADLYENPMGLMGFEFIEFASPTPGTLEPIFEIMGFTKVATHRSKNVHLYRQGAINLILNNEPHSVASYFAAEHGPSVCGMAFRVKDSQKAYNRALELGAQPIHIETGPMELNLPAIKGIGGAPLYLIDRFGEGSSIYDIDFVFLEGVDRNPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLTDDLVKTWDQLKKIGMRFMTAPPDTYYEMLEGRLPNHGEPVDQLQSRGILLDGASDKEDKRLLLQIFSETLMGPVFFEFIQRKGDDGFGPSNFKELFESIERDQVRRGVLATE

SEQ ID NO: 54 - protein, 358 aa, Artificial Sequence
  HPPD mutant - Axmi305H-Evo41
MADLYENPMGLMGFEFIEFASPTPGTLEPIFEIMGFTKVATHRSKNVHLYRQGAINLILNNEPHSVASYFAAEHGPSVCGMAFRVKDSQKAYNRALELGAQPIHIETGPMELNLPAIKGIGGAPLYLIDRFGEGSSIYDIDFVFLEGVDRNPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLTDDLVKTWDQLKKIGMRFMTAPPDTYYEMLEGRLPNHGEPVDQLQSRGILLDGASDKEDKRLLLQIFSETLMGPVFFEFIQRKGDDGFGPWNFAQLFESIERDQVRRGVLATE

SEQ ID NO: 55 - protein, 373 aa, Artificial Sequence
  HPPD mutant - Axmi305H-Evo40
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGVKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPSNFKELFETMELDQMRRGVLKT

SEQ ID NO: 56 - protein, 373 aa, Artificial Sequence
  HPPD mutant - Axmi305H-Evo41
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGVKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPWNFAQLFETMELDQMRRGVLKT

SEQ ID NO: 57 - protein, 357 aa, Pseudomonas aeruginosa
MNAVAKIEQHNPIGTDGFEFVEFTAPDAKGIEQLRQLFNMMGFTETAKHRSKEVFLFQQNDINIVLNGSPTGHVHEFALKHGPSACAMAFRVKNASQAAAYAESQGAKLVGSHANFGELNIPSLEGIGGSLLYLVDRYGDRSIYDVDFEFIEGRSANDNSVGLTYIDHLTHNVKRGQMDVWSGFYERIANFREIRYFDIEGKLTGLFSRAMTAPCGKIRIPINESADDTSQIEEFIREYHGEGIQHIALTTDDIYATVRKLRDNGVKFMSTPDTYYEKVDTRVAGHGEPLEQLRELNLLIDGAPGDDGILLQIFTDTVIGPIFFEIIQRKGNQGFGEGNFKALFESIEEDQIRRGVI

SEQ ID NO: 58 - protein, 358 aa, Pseudomonas agarici
MADLYENPMGLMGFEFIEFASPTPGTLEPIFEIMGFTKVATHRSKNVHLYRQGAINLILNNEPHSVASYFAAEHGPSVCGMAFRVKDSQKAYNRALELGAQPIHIETGPMELNLPAIKGIGGAPLYLIDRFGEGSSIYDIDFVFLEGVDRNPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLTDDLVKTWDQLKKIGMRFMTAPPDTYYEMLEGRLPNHGEPVDQLQSRGILLDGASDKEDKRLLLQIFSETLMGPVFFEFIQRKGDDGFGEGNFKALFESIERDQVRRGVLATE

SEQ ID NO: 59 - protein, 373 aa, Comamonas testosteroni
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGVKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGEGNFKALFETMELDQMRRGVLKT

SEQ ID NO: 60 - DNA, 1071 bp, Pseudomonas aeruginosa
atgaacgccgtggccaagatcgaacagcacaatcccatcggtaccgacggattcgaattcgtcgagttcaccgcccccgacgccaagggcatcgagcagctgcgccagctgttcaacatgatgggcttcaccgaaaccgccaagcatcgttccaaggaagtcttcctgttccagcagaacgatatcaacatcgtgctcaacggcagcccaaccgggcatgtccatgaattcgccctcaagcacggcccgagcgcctgcgccatggccttccgggtgaagaacgcttcccaggccgccgcctacgccgaatcccagggcgccaagctggtgggcagccacgccaacttcggcgagctgaacatcccttccctggaaggcatcggcggttcgctgctgtatcttgtcgaccgctacggcgaccgcagcatctatgacgtcgacttcgagttcatcgaaggccgcagcgccaacgacaactcggtcggcctgacctacatcgaccacctcacccacaacgtcaagcgcggccagatggacgtctggtccggtttctacgagcgcatcgccaacttccgcgagattcgctacttcgacatcgaaggcaagctcaccggcctgttctcccgcgccatgaccgcaccttgcgggaagatccgcatcccgatcaacgagtcggccgacgatacctcgcagatcgaggaattcatccgcgaataccatggcgaaggcatccagcacatcgccctgaccaccgacgacatctatgccaccgtgcgcaagctgcgcgacaacggcgtgaagttcatgtcgaccccggacacctactacgagaaggtcgacacccgcgtcgccgggcatggcgagccgctcgagcaactgcgcgaactgaacctgctgatcgacggcgccccgggcgacgacggcatcctgctgcagatcttcaccgacacggtgatcggcccgatcttcttcgagatcatccagcgcaagggcaaccagggcttcggcgagggcaatttcaaggccctgttcgagtccatcgaggaagaccagattcgccgcggcgtgatc

SEQ ID NO: 61 - DNA, 1074 bp, Pseudomonas agarici
atggcagatttatacgaaaacccaatgggcctgatgggcttcgagttcatcgagttcgcatcgccgactcctggcaccctggagccgatcttcgagatcatgggcttcaccaaggtcgcgacccaccgttccaagaacgtgcacctgtatcgccagggcgcgatcaacctgatcctcaacaacgaaccccacagcgttgcttcgtacttcgcggctgaacacggcccgtccgtttgcggcatggcgttccgggtcaaggattcgcagaaggcctacaaccgcgcactggaactcggcgcccagccgatccacatcgaaacaggcccgatggagctgaacctgccggcgatcaaaggcattggcggcgcgccgctgtacctgatcgaccgtttcggcgaaggcagctcgatctatgacatcgacttcgtgttcctcgaaggcgttgaccgcaacccggtcggtgccggcctgaagatcatcgaccacctgacccacaacgtgtatcgcggccgcatggcctactgggccaacttctacgagaagctgttcaacttccgcgagatccgctacttcgacatcaaaggcgaatacaccggcctgacctcgaaagcgatgaccgcaccggacggcatgatccgcatcccgctcaacgaagaatcgtcgaagggtgccgggcagatcgaagagttcctgatgcagttcaacggcgaaggcatccagcacgtggcgttcctcaccgacgacctggtcaagacctgggatcagttgaagaagatcggcatgcgtttcatgaccgcgccgccggacacctactacgaaatgctcgaaggccgcctgccgaaccacggcgagccggtggatcaactgcaatcgcgcggcatcctgctcgacggtgcgtcggataaagaagacaagcgtctgctgctgcagatcttctcggaaaccctgatgggcccggtgttcttcgaattcatccagcgtaaaggcgatgatggtttcggagaaggcaacttcaaggctctgttcgaatcgatcgagcgtgaccaggtgcgtcgtggcgtgctcgctaccgag

SEQ ID NO: 62 - DNA, 1122 bp, Comamonas testosteroni
atgaacgccccgttgacccaaagcaatgccagccagttccagacctgggacaaccccatgggcacggacggcttcgagttcgtcgaatacgcggcccccgatcccgtggccatgggtcagctgttcgagcgcatgggctttcaggccattgccaagcaccgccgcaagaacgtgaccctgtatcgccagggcgagatcaacttcatcatcaatgccgaacccgacagctttgcccagcgtttcgcgcgtctgcacggccccagcgtctgcgccatcgccatccgcgtcaacgacgccaagtacgcctatgagcgcgccacctcgctgggtgcctggggctatgcccagcaggccgcccccggcgaactgagcattcccgccatcaagggcattggcgactccctgatctatttcatcgacaaatggcgcggcaagaatggcgccaaggacggtgatctcggcaatatcagcttcttcgacgtggacttcgagcctctgcccggtgccgatctgcatcccgagggcctgggcctgacctatatcgaccacctgaccaacaacgtctaccgcggccgcatggccgagctggccgagttctacgagcgcatcttcaacttccgcgagatccgctacttcgacatcgaaggccaggccacaggcgtcaagagcaaggccatgaccagcccctgcggcaagatccgcattcccatcaacgaggaaggcaacgacaaggccggccagattcaggagtatctggacatgtaccgcggcgaaggcatacagcacatcgcgctgggatcgaccaatctctacgacaccgtggacggtctgcagatgaacggcatcaagctgctgaacaccagcgagacctattacgagctgctgcccaagcgcatcccggacctgcaggaacccattcccgagctgctggcgcgcaacatccttgtggacggccagcccggcgagctgctgctgcagatcttcagcgaaaaccagctgggtcccatcttcttcgagttcatccagcgcaagggcaatagcggctttggcgagggcaatttcaaggccttgttcgagaccatggaactcgaccagatgcgccgcggcgtgctcaagacctga

SEQ ID NO: 63 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 64 - protein, 439 aa, Artificial Sequence
  HPPD mutant - Avena sativum deletion mutant
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 65 - protein, 444 aa, Zea mays
MGPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEDAFRASVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAAAAQGS

SEQ ID NO: 66 - protein, 445 aa, Arabidopsis thaliana
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 67 - protein, 434 aa, Hordeum vulgare
MPPTPTTPAATGAAAAVTPEHARPHRMVRFNPRSDRFHTLSFHHVEFWCADAASAAGRFAFALGAPLAARSDLSTGNSAHASQLLRSGSLAFLFTAPYANGCDAATASLPSFSADAARRFSADHGIAVRSVALRVADAAEAFRASRRRGARPAFAPVDLGRGFAFAEVELYGDVVLRFVSHPDGTDVPFLPGFEGVTNPDAVDYGLTRFDHVVGNVPELAPAAAYIAGFTGFHEFAEFTAEDVGTTESGLNSVVLANNSEGVLLPLNEPVHGTKRRSQIQTFLEHHGGPGVQHIAVASSDVLRTLRKMRARSAMGGFDFLPPPLPKYYEGVRRLAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 68 - protein, 442 aa, Daucus carota
MGKKQSEAEILSSNSSNTSPATFKLVGFNNFVRANPKSDHFAVKRFHHIEFWCGDATNTSRRFSWGLGMPLVAKSDLSTGNSVHASYLVRSANLSFVFTAPYSPSTTTSSGSAAIPSFSASGFHSFAAKHGLAVRAIALEVADVAAAFEASVARGARPASAPVELDDQAWLAEVELYGDVVLRFVSFGREEGLFLPGFEAVEGTASFPDLDYGIRRLDHAVGNVTELGPVVEYIKGFTGFHEFAEFTAEDVGTLESGLNSVVLANNEEMVLLPLNEPVYGTKRKSQIQTYLEHNEGAGVQHLALVSEDIFRTLREMRKRSCLGGFEFMPSPPPTYYKNLKNRVGDVLSDEQIKECEDLGILVDRDDQGTLLQIFTKPVGDRPTLFIEIIQRVGCMLKDDAGQMYQKGGCGGFGKGNFSELFKSIEEYEKTLEAKQITGSAAA

SEQ ID NO: 69 - protein, 380 aa, Streptomyces avermitilis
MTQTTHHTPDTARQADPFPVKGMDAVVFAVGNAKQAAHYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCVGNVELGRMNEWVGFYNKVMGFTNMKEFVGDDIATEYSALMSKVVADGTLKVKFPINEPALAKKKSQIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKALFEAIEREQEKRGNL

SEQ ID NO: 70 - protein, 419 aa, Mycosphaerella graminicola
MAPGALLVTSQNGRTSPLYDSDGYVPAPAALVVGGEVNYRGYHHAEWWVGNAKQVAQFYITRMGFEPVAHKGLETGSRFFASHVVQNNGVRFVFTSPVRSSARQTLKAAPLADQARLDEMYDHLDKHGDGVKDVAFEVDDVLAVYENAVANGAESVSSPHTDSCDEGDVISAAIKTYGDTTHTFIQRTTYTGPFLPGYRSCTTVDSANKFLPPVNLEAIDHCVGNQDWDEMSDACDFYERCLGFHRFWSVDDKDICTEFSALKSIVMSSPNQVVKMPINEPAHGKKKSQIEEYVDFYNGPGVQHIALRTPNIIEAVSNLRSRGVEFISVPDTYYENMRLRLKAAGMKLEESFDIIQKLNILIDFDEGGYLLQLFTKPLMDRPTVFIEIIQRNNFDGFGAGNFKSLFEAIEREQDLRGNL

SEQ ID NO: 71 - protein, 399 aa, Coccicoides immitis
MAPAADSPTLQPAQPSDLNQYRGYDHVHWYVGNAKQAATYYVTRMGFERVAYRGLETGSKAVASHVVRNGNITFILTSPLRSVEQASRFPEDEALLKEIHAHLERHGDGVKDVAFEVDCVESVFSAAVRNGAEVVSDVRTVEDEDGQIKMATIRTYGETTHTLIERSGYRGGFMPGYRMESNADATSKFLPKVVLERIDHCVGNQDWDEMERVCDYYEKILGFHRFWSVDDKDICTEFSALKSIVMASPNDIVKMPINEPAKGKKQSQIEEYVDFYNGAGVQHIALRTNNIIDAITNLKARGTEFIKVPETYYEDMKIRLKRQGLVLDEDFETLKSLDILIDFDENGYLLQLFTKHLMDRPTVFIEIIQRNNFSGFGAGNFRALFEAIEREQALRGTLI

SEQ ID NO: 72 - protein, 350 aa, Synechococcoideae spp.
MNPSIRIVQGIHHLHFYLWDLPRWREHFCRVWGFRVASDAGNTLELEQGSLRLRLSQPARAGDEVDRHLQRHGPGVVDVALAVGEQELPALAELLRGRGAQLAWIPAAAALCLHTPYGIRHSLIPGPLDAAPAEAGLFSHWDHVVLNVEQGSLQAAADWYGRVLGWRRLYRYSIGTATSGLESVVVGDPEAGIQWAINEPTCAASQIQEFLHAHGGPGIQHAALHSSDIVASLRRLRQGGVDFLQVAPQYYTSLERELGLALRSALGQAISWQDLVEQQILLDATLPASDGQDRPLLLQTFTQPLFGRPTFFFEVIQRLGGATGFGEANFQALFEALERQQRQRHQALTP

SEQ ID NO: 73 - protein, 368 aa, Picrophilus torridus
MYGKNLISELREKEIFKRLHHVEFYVSSAKTWSYFMNRGLGFKTVAYAGPETGIRDKISYVMSQGTARISFTSSMNDDSYISNHVKKHGDGVKDIALEVDDLDEAKSLIEKYGTKVSKINEIKDGNGKIRTAEIKTYGETVHTLIETGDYNGVFMPGYEESEINSKNTGIKKIDHIVGNVYEGEMDSWVNFYIEKLGFEHLITFDDKDIRTDYSALRSKVVKYNDDIVFPINEPAKGLRKSQIEEYLDYYRSEGVQHIALLTDDIIKTVSMMEENGIEFLKTPGSYYESLSSRIGSIDEDLNEIEKHNILVDRDENGYLLQIFTKPVTDRPTFFFEVIQRKGARSFGNGNFKALFEAIEREQAKRGNL

SEQ ID NO: 74 - protein, 387 aa, Kordia algicida
MAAEIKNLKDLQNTEYGLKKLFDEAEDFLPLLGTDYVELYVGNAKQSAHFYKTAFGFQSEAYAGLETGLTDRVSYVLKQDKIRLVLTTPLGKGGEINEHIDLHGDGVKVVALWVEDATKAFEETTKRGAKPYMEPTKEEDENGYVIRSGIYTYGETVHVFVERKNYNGVFLPGYQRWESHYNPEPVGLKFIDHMVGNVGWGEMKEWCEFYAKVMGFAQIISFTDDDISTDFTALMSKVMSNGNGRIKFPINEPAEGKKKSQIEEYLDFYNGSGVQHIAVATDNIIDTVSQMRERGVEFLYVPDTYYDDLLERVGDIDEDVEELKKHGILIDRDEEGYLLQLFTKTIVDRPTMFFEVIQRKGAQSFGVGNFKALFEAIEREQAARGTL

SEQ ID NO: 75 - protein, 382 aa, Blepharisma japonicum
MTYYDKQETRPDLGEFYGFHHVRFYVSNSEQAASFYTSRFGFSPVAYEGLETGNQKFCTNVVRSNHVVIAFTSALTPEDNEVNRHVGKHSDGVQDIAFSVSDARGMYEKAIAKGCKSFREPQVLQDQFGSVIIASLQTYGDTVHTLVQNVDYTGPFLPGFRAITKDDPLNSAFPQVNYDIIDHVVGNQPGGDMTPTVEWYEKYLEFHRYWSADESVIHTDYSALRSVVVADWDEVIKMPINEPADGLRKSQIQEYVEYYGGAGVQHIALKVNDIISVISTLRARGVEFLEVPPKYYDSLRKRLAHSAVQIEEDLKRIEDLHILVDFDDRGYLLQIFTKPVEDRPTLFYEIIQRHNNNGFGIGNFKALFESLEQEQERRGNLI

SEQ ID NO: 76 - protein, 401 aa, Rhodococcus sp.
MTIEQTLTDKERLAGLDLGQLEQLVGLVEYDGTRDPFPVSGWDAVVWVVGNATQTAHYFQSAFGMTLVAYSGPTTGNRDHHSFVLESGAVRFVIKGAVNPDSPLIDHHRTHGDGVVDIALAVPDVDKCIAHARAQGATVLDEPHDVTDDHGTVRLAAIATYGDTRHTLVDRSHYTGPYLPGYTARTSGHTKRDGAPKRLFQALDHVVGNVELGKMDHWVDFYNRVMGFTNMAEFVGEDIATDYSALMSKVVSNGNHRVKFPLNEPALAKKRSQIDEYLDFYRGPGAQHLALATNDILTAVDQLTAEGVEFLATPDSYYEDPELRARIGNVRAPIAELQKRGILVDRDEDGYLLQIFTKPLVDRPTVFFELIERHGSLGFGIGNFKALFEAIEREQAARGNF

SEQ ID NO: 77 - DNA, 18 bp, Artificial Sequence
  histidine tag
catcaccatcaccatcac

SEQ ID NO: 78 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGVKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGEYNFKGLFETMELDQMRRGVLKT

SEQ ID NO: 79 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGVKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGEYNFGGLFETMELDQMRRGVLKT

SEQ ID NO: 80 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREARYFDIEGQATGVKSKAMTSPCGKIRIPINEEGNDKAGHIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPYNFKGLFETMELDQMRRGVLKT

SEQ ID NO: 81 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREARYFDIEGQATGIKSKAMTSPCGKIRIPINEEGNDKAGHIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPYNFKGLFETMELDQMRRGVLKT

SEQ ID NO: 82 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRGAELAEFYERIFNFREIRYFDIEGQATGVKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPYNFKGLFETMELDQMRRGVLKT

SEQ ID NO: 83 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGVKSKAMTSPCGKIRIPINEEGNDKAGHIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPYNFKGLFETMELDQMRRGVLKT

SEQ ID NO: 84 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGIKSKAMTSPCGKIRIPINEEGNDKAGQIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPYNFKGLFETMELDQMRRGVLKT

SEQ ID NO: 85 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREIRYFDIEGQATGIKSKAMTSPCGKIRIPINEEGNDKAGHIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGPYNFKGLFETMELDQMRRGVLKT

SEQ ID NO: 86 - protein, 373 aa, Artificial Sequence
  variant HPPD
MNAPLTQSNASQFQTWDNPMGTDGFEFVEYAAPDPVAMGQLFERMGFQAIAKHRRKNVTLYRQGEINFIINAEPDSFAQRFARLHGPSVCAIAIRVNDAKYAYERATSLGAWGYAQQAAPGELSIPAIKGIGDSLIYFIDKWRGKNGAKDGDLGNISFFDVDFEPLPGADLHPEGLGLTYIDHLTNNVYRGRMAELAEFYERIFNFREARYFDIEGQATGSKSKAMTSPCGKIRIPINEEGNDKAGHIQEYLDMYRGEGIQHIALGSTNLYDTVDGLQMNGIKLLNTSETYYELLPKRIPDLQEPIPELLARNILVDGQPGELLLQIFSENQLGPIFFEFIQRKGNSGFGAYNFKALFETMELDQMRRGVLKT

SEQ ID NO: 87 - protein, 373 aa, Artificial Sequence; identical to SEQ ID NO: 56
  variant HPPD

SEQ ID NO: 88 - protein, 358 aa, Artificial Sequence
  variant HPPD
MADLYENPMGLMGFEFIEFASPTPGTLEPIFEIMGFTKVATHRSKNVHLYRQGAINLILNNEPHSVASYFAAEHGPSVCGMAFRVKDSQKAYNRALELGAQPIHIETGPMELNLPAIKGIGGAPLYLIDRFGEGSSIYDIDFVFLEGVDRNPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLTDDLVKTWDQLKKIGMRFMTAPPDTYYEMLEGRLPNHGEPVDQLQSRGILLDGASDKEDKRLLLQIFSETLMGPVFFEFIQRKGDDGFGPWNFKGLFESIERDQVRRGVLATE

SEQ ID NO: 89 - DNA, 2047 bp, Glycine max
ttacagtcactatcatatttctatgacggcctaaccattttttctttatccaagcaaagatcgtagaataattttaattttattagaagaatatattatctttattaagcctattaattaagagataacaggaagcattggatagcattctttctttgaaaaaaatggaatcaatcggggtgttaatgacttgcccaatgcacagttacatccaagaagagctcgcaaaacgcttcaacctcttcaaactctggcactatccctcgttctccgccttcgcccaagcccacgcccattccattcgcgccctcgtcgcaagcgccaaagtcggagtcgacgccgccaccatcgattcccttcccaacctcgagattgtctccacctacagcgtcggatacgacaacatcgacctccacaaatgcagacacagagcaatccccgtcaccaacacgcccaacgttttaaccgacgacgtggccgacgtagccatcgccctcgccttgtccctcctctgcagaatttgtccccgaaattccacttggcaattcacaccaaagcttagtggtaaagcggttggaatagttgggctgggaaggattggttgggcgattgcgaagagagccgagggttttgggtgtccagtgagttaccattccagatctgaaaaatcagagacagggtataagtattactctcacatcattgatttggcggctaactctgaagtgctctttgtggcgtgtacccttagtgaagaaacgcgtcacattgtgaaccgtggggttattgatgcgttgggcccgaaagggattctgatcaatgttgggcgaggcccgcacgtggatgagcccgaactggtggccgcgttgattgaagggagactaggtggggcgggccttgatgtgtttgagaacgagccggaggtgcctgaagacctgctggggcttgagaatcttgtcatgacccctcatgtggggactgacactctggaaacttgcatcgctatgggagaccttgtaattgctaacttagaggcacactttcttggcaacccacttttcacacctgtcctttagatcgctggctactaccttgccagagctcagttacatttcaatttttctacttcagtgagttgtggataacagaaaataaaaataaaaaatcatccattccacttcctgttcacaaaatgcacatcacgaattcattctagataaacacatcccaagttatttgtatcaagcgcacaaataaccacccttaaacgatatgaattcatatagaagagacaatacatagattaagcatcttcatatgccatccggataacatagaccaatagtatgtgcattgattacttaaactaaaatcttgccccgaccaaattttttcagaaatgaaccacgacattggacgggtggcaatgaaaaaggtgcagtgaagatgcaacatctagtagatgttatcgtccaatagatggtcatatggaagattgaccacgagagggctggtagtggcggtgacagtgtacatgatgaggaggaggaagctgaagcttgcgatgacatggaagctgacgacgtctgaaggaggataacataataggaaaaggaggggcagggattgtgtaccgcgggtccatgcaaacggaacttggaggcccatgttgctgattttggccttgccaagttcttgtacgaccctggcgcctctcagtccatgtcctccattgctggctcctacggctacattgctccaggggaggggcaacaagagtagtggagacaattttcttgctgctccaaacattgatcattcacaggactttgtgagaaaggatcttaaagaatggttatgctggatgaggaacaattttgtggagagattgagtcacatttatggaatgatttcaagccaaaacttcataggaggtgatcattgtatgacgttaaaggaaacagacatagagtaagtgtaaatctgattggagaacaactttttatgacacgaaattgatttgaactgaggatgttttataatttca

SEQ ID NO: 90 - DNA, 1235 bp, Glycine max
gtctgactccgagtctgtctttgttgcgtttcgagtgtctgtggaatccctaaccctaatttattgtgtggcgaaatgagatcaattggggtactcctggtggctcaggtgataccgtacctggagcaagagctggacaagcgctacaagctgtttcgggcgtatgatcagccgcagacggcgcaggttctaagccagcacgcgagctcgatccgtgcggtggtcggaaactcgaacgccggcgccgacgcggagctgatcgaggcgctgccgaagctggagatcgtgtcgagcttcagcgtgggagtggacaggatagacctggacaggtgcaaggagaaaggaattcgcgtcaccaacacgccggacgtgctgaccgatgaagtcgccgacctcgctatcggattgatgctggcgctgctcaggaggatctgcgagtgcgatcgttatgtcaggagcggcaagtggaagaaaggggactacaaactgaccactaagttctctgggaaaactgttggcattattgggctagggaggattggtcaagcaattgctaagagagctgaaggattcaactgccccatatgctactactctagaactcaaaaaagagactcaaactacaagtactatcctagtgttgtagaactggcatctaactgcgacatactggtagttgcttgcccactgacggaggaaactcatcacatcatcaacagggaggtgatcaatgcactgggtcccaagggttatcttattaacattggacgaggcaagcatgttgatgaggcagagttagtgccagctctgctagaaggtcgtttgggtggtgctgggctagatgtgtttgaaaatgagcctactgttccagaagagctatttgggcttgaaaatgttgtcttgttgcctcatgtcggaagtggcacaatagaaactcgaactgccatggctgaccttgtccttggaaacctagacgctcatttccttggaaatccactgttaacacccttggtttaatcaatatgccatcatggaactaccaggactgattgtccccctgcattactaccgtttaataactttttgtgacatggaaattgatcattgtaagagcttcttcatatttgtgctggtgcttaatctattgtaaacaatgatttagttcaccttcattgtgacatcttaataagattatcaatcattcaatattgatcgacaaaaaaaaaaaaaaaaaa

SEQ ID NO: 91 - DNA, 608 bp, Artificial Sequence
  hdrGm-hpf1 concatemer
ttagtggtaaagcggttggaatagttgggctgggaaggattggttgggcgattgcgaagagagccgagggttttgggtgtccagtgagttaccattccagatctgaaaaatcagagacagggtataagtattactctcacatcattgatttggcggctaactctgaagtgctctttgtggcgtgtacccttagtgaagaaacgcgtcacattgtgaaccgtggggttattgatgcgttgggcccgaaagggattctgatcaatgttgggcgaggcccgcacgtggatgagcccgaactggtggccgcaaactgttggcattattgggctagggaggattggtcaagcaattgctaagagagctgaaggattcaactgccccatatgctactactctagaactcaaaaaagagactcaaactacaagtactatcctagtgttgtagaactggcatctaactgcgacatactggtagttgcttgcccactgacggaggaaactcatcacatcatcaacagggaggtgatcaatgcactgggtcccaagggttatcttattaacattggacgaggcaagcatgttgatgaggcagagttagtgccagctcg

SEQ ID NO: 92 - protein, 303 aa, Glycine max
MESIGVLMTCPMHSYIQEELAKRFNLFKLWHYPSFSAFAQAHAHSIRALVASAKVGVDAATIDSLPNLEIVSTYSVGYDNIDLHKCRHRAIPVTNTPNVLTDDVADVAIALALSLLCRICPRNSTWQFTPKLSGKAVGIVGLGRIGWAIAKRAEGFGCPVSYHSRSEKSETGYKYYSHIIDLAANSEVLFVACTLSEETRHIVNRGVIDALGPKGILINVGRGPHVDEPELVAALIEGRLGGAGLDVFENEPEVPEDLLGLENLVMTPHVGTDTLETCIAMGDLVIANLEAHFLGNPLFTPVL

SEQ ID NO: 93 - protein, 313 aa, Glycine max
MRSIGVLLVAQVIPYLEQELDKRYKLFRAYDQPQTAQVLSQHASSIRAVVGNSNAGADAELIEALPKLEIVSSFSVGVDRIDLDRCKEKGIRVTNTPDVLTDEVADLAIGLMLALLRRICECDRYVRSGKWKKGDYKLTTKFSGKTVGIIGLGRIGQAIAKRAEGFNCPICYYSRTQKRDSNYKYYPSVVELASNCDILVVACPLTEETHHIINREVINALGPKGYLINIGRGKHVDEAELVPALLEGRLGGAGLDVFENEPTVPEELFGLENVVLLPHVGSGTIETRTAMADLVLGNLDAHFLGNPLLTPLV

SEQ ID NO: 94 - DNA, 85 bp, Artificial Sequence
  intron hdrGm
gtttttaatttaatttttatcctcaagaattctattttcatattctcagttggatgttacataatttgcgtgtttttatatttag

(19) United States
(12) Patent Application Publication
(10) Pub. No.: US 2021/0032651 A1
(43) Pub. Date: Feb. 4, 2021

(54) IMPROVEMENT OF HERBICIDE TOLERANCE TO HPPD INHIBITORS BY DOWN-REGULATION OF PUTATIVE 4-HYDROXYPHENYLPYRUVATE REDUCTASES IN SOYBEAN

(71) Applicant: BASF SE, Ludwigshafen Am Rhein (DE)

(72) Inventor: Manuel DUBALD, Research Triangle Park, NC (US)

(21) Appl. No.: 16/758,836

(22) PCT Filed: Oct. 18, 2018
(86) PCT No.: PCT/US2018/056459

§ 371 Date: Apr. 23, 2020

Related U.S. Application Data
(60) Provisional application No. 62/576,564, filed on Oct. 24, 2017.

(51) Int. Cl. C12N 15/82; C12N 9/04; C12N 9/02

(52) U.S. Cl. CPC C12N 15/8274; C12N 9/0006; C12Y 113/11027; C12Y 101/01237; C12N 9/0069

(57) ABSTRACT

A method for conferring tolerance to a 4-hydroxyphenylpyruvate dioxygenase (HPPD) inhibitor herbicide in a plant, comprising reducing expression of at least one 4-hydroxyphenylpyruvate reductase (HPPR) enzyme in the plant.

### FIELD OF THE INVENTION

This invention relates to plant molecular biology, particularly down-regulation of putative 4-hydroxyphenylpyruvate (HPP) reductases that confer improved tolerance to 4-hydroxyphenylpyruvate dioxygenase (HPPD) inhibitor herbicides in soybean.

### BACKGROUND OF THE INVENTION

The 4-hydroxyphenylpyruvate dioxygenases (HPPDs) are enzymes which catalyze the reaction in which para-hydroxyphenylpyruvate (abbreviated herein as HPP), a tyrosine degradation product, is transformed into homogentisate (abbreviated herein as HG), the precursor in plants of tocopherol and plastoquinone (Crouch N. P. et al. (1997), Tetrahedron, 53, 20, 6993-7010, Fritze et al. (2004), Plant Physiology 134:1388-1400). Tocopherol acts as a membrane-associated antioxidant. Plastoquinone, firstly acts as an electron carrier between PSII and the cytochrome b6/f complex and secondly, is a redox cofactor for phytoene desaturase, which is involved in the biosynthesis of carotenoids.

Up to now, more than 1000 nucleic acid sequences from various organisms present in the NCBI database were annotated as coding for a putative protein having an HPPD domain. But for most of those, it has not been proven that the protein would have an HPPD enzymatic activity either in an in vitro assay or in an in planta approach, nor that such HPPD protein can confer herbicide tolerance to HPPD inhibitor herbicides when expressed in a plant. Several HPPD proteins and their primary sequences have been described in the state of the art, in particular the HPPD proteins of bacteria such as Pseudomonas (Rüetschi et al., Eur. J. Biochem., 205, 459-466, 1992, WO96/38567), Kordia (WO2011/076889) Synechococcus (WO2011/076877), and Rhodococcus (WO2011/076892), of protists such as Blepharisma (WO2011/076882), of euryarchaeota such as Picrophilus (WO2011/076885) of plants such as Arabidopsis (WO96/38567, GENBANK® AF047834), carrot (WO 96/38567, GENBANK® 87257), Avena sativa (WO2002/046387, WO2011/068567), wheat (WO2002/046387), Brachiaria platyphylla (WO2002/046387), Cenchrus echinatus (WO2002/046387), Lolium rigidum (WO2002/046387), Festuca arundinacea (WO2002/046387), Setaria faberi (WO 2002/046387), Eleusine indica (WO2002/046387), Sorghum (WO2002/046387, WO2012/021785), corn (WO2012/021785), Coccicoides (GENBANK® COITRP), of Coptis japonica (WO2006/132270), Chlamydomonas reinhardtii (ES 2275365; WO2011/145015), or of mammals such as mouse or pig.

Inhibition of HPPD leads to uncoupling of photosynthesis, deficiency in accessory light-harvesting pigments and, most importantly, to destruction of chlorophyll by UV-radiation and reactive oxygen species (bleaching) due to the lack of photo protection normally provided by carotenoids (Norris et al. (1995), Plant Cell 7: 2139-2149). Bleaching of photosynthetically active tissues leads to growth inhibition and plant death.

Some molecules which inhibit HPPD, and which inhibit transformation of the HPP into homogentisate while binding specifically to the enzyme, have proven to be very effective herbicides.

At present, most commercially available HPPD inhibitor herbicides belong to one of these chemical families:
1) the triketones, e.g. sulcotrione [i.e. 2-[2-chloro-4-(methylsulfonyl)benzoyl]-1,3-cyclohexanedione], mesotrione [i.e. 2-[4-(methylsulfonyl)-2-nitrobenzoyl]-1,3-cyclohexanedione]; tembotrione [i.e. 2-[2-chloro-4-(methylsulfonyl)-3-[(2,2,2,-tri-fluoroethoxy)methyl] benzoyl]-1,3-cyclo-hexanedione]; tefuryltrione [i.e. 2-[2-chloro-4-(methylsulfonyl)-3-[[(tetrahydro-2-furanyl)methoxy]methyl]benzoyl]-1,3-cyclohexanedione]]; bicyclopyrone [i.e. 4-hydroxy-3-[[2-[(2-methoxyethoxy)methyl]-6-(trifluoromethyl)-3-pyridinyl]carbonyl]bicyclo[3.2.1]oct-3-en-2-one]; Benzobicyclon [i.e. 3-(2-chloro-4-mesylbenzoyl)-2-phenylthiobicyclo[3.2.1]oct-2-en-4-one];
2) the diketonitriles, e.g. 2-cyano-3-cyclopropyl-1-(2-methylsulphonyl-4-trifluoromethylphenyl)-propane-1,3-dione and 2-cyano-1-[4-(methylsulphonyl)-2-trifluoromethylphenyl]-3-(1-methylcyclopropyl)propane-1,3-dione;
3) the isoxazoles, e.g. isoxaflutole [i.e. (5-cyclopropyl-4-isoxazolyl)[2-(methylsulfonyl)-4-(trifluoromethyl)phenyl]methanone]. In plants, isoxaflutole is rapidly metabolized in DKN, a diketonitrile compound which exhibits the HPPD inhibitor property;
4) the pyrazolinates, e.g. topramezone [i.e. [3-(4,5-dihydro-3-isoxazolyl)-2-methyl-4-(methylsulfonyl) phenyl](5-hydroxy-1-methyl-1H-pyrazol-4-yl)methanone], and pyrasulfotole [i.e. (5-hydroxy-1,3-dimethylpyrazol-4-yl(2-mesyl-4-trifluaromethylphenyl)methanone]; pyrazofen [i.e. 2-[4-(2,4-dichlorobenzoyl)-1,3-dimethylpyrazol-5-yloxy]acetophenone];
5) N (1,2,5-oxadiazol-3-yl)benzamides (WO2011/035874) and N-(1,3,4-oxadiazol-2-yl)benzamides (WO2012/126932), e.g. 2-methyl-N-(5-methyl-1,3,4-oxadiazol-2-yl)-3-(methylsulfonyl)-4-(trifluoromethyl)benzamide (hereinafter also named “Cmpd. 1”);
6) N-(tetrazol-5-yl)- or N-(triazol-3-yl)arylcarboxamides (WO2012/028579), e.g. 2-chloro-3-ethoxy-4-(methylsulfonyl)-N-(1-methyl-1H-tetrazol-5-yl)benzamide (hereinafter also named “Cmpd.2”); 4-(difluoromethyl)-2-methoxy-3-(methylsulfonyl)-N-(1-methyl-1H-tetrazol-5-yl)benzamide (hereinafter also named “Cmpd. 3”); 2-chloro-3-(methylsulfanyl)-N-(1-methyl-1H-tetrazol-5-yl)-4-(trifluoromethyl)benzamide (hereinafter also named “Cmpd. 4”); 2-(methoxymethyl)-3-(methylsulfinyl)-N-(1-methyl-1H-tetrazol-5-yl)-4-(trifluoromethyl)benzamide (hereinafter also named “Cmpd. 5”);
7) Pyridazinone derivatives as described in WO2013/050421 and WO2013/083774;
8) Substituted 1,2,5-oxadiazoles as described in WO2013/072300 and WO2013/072402; and
9) Oxoprazin derivatives as described in WO2013/054495.

These HPPD inhibitor herbicides can be used against grass and/or broad leaf weeds in fields of crop plants that display metabolic tolerance, such as maize (Zea mays), rice (Oryza Sativa) and wheat (Triticum aestivum) in which they are rapidly degraded (Schulz et al. (1993), FEBS letters, 318, 162-166; Mitchell et al. (2001), Pest Management Science, Vol 57, 120-128; Garcia et al. (2000), Biochem., 39, 7501-7507; Pallett et al. (2001), Pest Management Science, Vol 57, 133-142). In order to extend the scope of use of these HPPD inhibitor herbicides, several efforts have been developed in order to confer to plants, particularly plants without or with an underperforming metabolic tolerance, a tolerance level acceptable under agronomic field conditions.

Besides the attempt of by-passing HPPD-mediated production of homogentisate (U.S. Pat. No. 6,812,010), overexpressing the sensitive enzyme so as to produce quantities of the target enzyme in the plant which are sufficient in relation to the herbicide has been performed (WO96/38567). Overexpression of HPPD resulted in better pre-emergence tolerance to the diketonitrile derivative (DKN) of isoxaflutole (IFT), but the tolerance level was not sufficient for tolerance to post-emergence treatment (Matringe et al. (2005), Pest Management Science 61: 269-276).

A third strategy was to mutate the HPPD in order to obtain a target enzyme which, while retaining its properties of catalyzing the transformation of HPP into homogentisate, was less sensitive to HPPD inhibitors than the native HPPD before mutation.

This strategy has been successfully applied for the production of plants tolerant to 2-cyano-3-cyclopropyl-1-(2-methylsulphonyl-4-trifluoromethylphenyl)-propane-1,3-dione and to 2-cyano-1-[4-(methylsulphonyl)-2-trifluoromethylphenyl]-3-(1-methylcyclopropyl)propane-1,3-dione (EP496630), two HPPD inhibitor herbicides belonging to the diketonitriles family (WO99/24585). Pro215Leu, Gly336Glu, Gly336Ile, and more particularly Gly336Trp (positions of the mutated amino acid are indicated with reference to the Pseudomonas fluorescens HPPD) were identified as mutations which are responsible for an increased tolerance to treatment with these diketonitrile herbicides.

More recently, introduction of a Pseudomonas fluorescens HPPD gene into the plastid genome of tobacco and soybean was shown to be more effective than nuclear transformation, conferring tolerance to post-emergence application of isoxaflutole (Dufourmantel et al. (2007), Plant Biotechnol J.5(1):118-33).

In WO2004/024928, the inventors sought to increase the prenylquinone biosynthesis (e.g., synthesis of plastoquinones, tocopherols) in the cells of plants by increasing the flux of the HPP precursor into the cells of these plants. This was done by connecting the synthesis of said precursor to the “shikimate” pathway by overexpression of a prephenate dehydrogenase (PDH) enzyme. They also noted that the transformation of plants with a gene encoding a PDH enzyme and a gene encoding an HPPD enzyme makes it possible to increase the tolerance of said plants to HPPD inhibitors.

In WO2009/144079, nucleic acid sequences encoding a HPPD with specific mutations at position 336 of the Pseudomonas fluorescens HPPD protein and their use for obtaining plants which are tolerant to HPPD inhibitor herbicides was disclosed.

In WO2002/046387, several domains of HPPD proteins originating from plants were identified that may be relevant to confer tolerance to various HPPD inhibitor herbicides, but neither in planta nor biochemical data were shown to confirm the impact of the as described domain functions.

In WO2008/150473, the combination of two distinct tolerance mechanisms—a modified Avena sativa gene coding for a mutant HPPD enzyme and a CYP450 Maize monooxygenase (nsf1 gene)—was exemplified in order to obtain an improved tolerance to HPPD inhibitor herbicides, but no data were disclosed demonstrating the synergistic effects based on the combination of both proteins.

Further, in US2011/0173718, a method to generate plants tolerant to HPPD inhibitors by overexpressing not only a gene coding for a tolerant HPPD, as for example from Avena sativa, but also in combination with several plant genes coding for an HST (homogentisate solanesyltransferase) protein was disclosed. However, the level of tolerance to selected HPPD inhibitor herbicides was rather limited.

In WO2011/094199 and US2011/0185444, the tolerance of several hundreds of soybean wild type lines to the HPPD inhibitor isoxaflutole was evaluated. Very few lines displayed reasonable levels of tolerance to the herbicides. The putative QTL (quantitative trait loci) responsible for the tolerance was identified. In this region of the genome, a gene coding for an ABC transporter was identified as being the main trait responsible for the improved tolerance to the HPPD inhibitor herbicide observed. However, transgenic plants expressing the identified genes did not display any improvement in tolerance to the tested HPPD inhibitor herbicides.

In WO2010/085705, several mutants of the Avena sativa HPPD were disclosed. It was shown that some of the variants displayed improved tolerance in vitro to the triketone “mesotrione”, however, only very few mutants were expressed in tobacco plants. Additionally, none of the tobacco plants expressing these mutants displayed improved tolerance to mesotrione or isoxaflutole compared to tobacco plants expressing the wild type Avena sativa HPPD gene.

US 2012/0042413 describes polypeptides having HPPD activity but also showing a certain insensitivity to at least one HPPD inhibitor and further suggests a certain set of mutations at different positions of HPPD enzymes and finally discloses biochemical data, as well as tolerance levels, of plants containing few of such mutated HPPDs. In EP 2453012, several mutants of HPPD were described; however, the improved tolerance of the described mutants was not demonstrated in planta against several HPPD inhibitor herbicides.

WO2014/043435 describes mutant HPPD enzymes derived from the native Pseudomonas fluorescens HPPD nucleotide sequence (Pf-HPPD, 1077 bp, as described in WO2009/144079) having HPPD activity with broad tolerance to HPPD inhibitor herbicides as demonstrated by biochemical data and tolerance levels of plant containing several of the disclosed Pf-HPPD mutant enzymes.

Hydroxyphenylpyruvate reductase (HPPR) is an enzyme that uses the same substrate as HPPD. It has been biochemically characterized only in a few plant species (e.g., Labiae, Colueus blumei). This pathway leads to secondary metabolites such as rosmarinic acid. It is unknown if this pathway exists in most plant species, but two genes annotated as HPPR(s) are reported in the soybean genome.

### SUMMARY OF THE INVENTION

Compositions and methods for conferring tolerance to HPPD inhibitor herbicides are provided.

Disclosed herein is a double-stranded ribonucleic acid (dsRNA) comprising a sense region with at least 94% sequence identity to a portion of at least 19 consecutive nucleotides of one or more endogenous HPPR gene(s) and an antisense region comprising a second sequence complementary to said sense region. In a preferred embodiment, a dsRNA comprising a sense region with at least 94% sequence identity to a portion of at least 19 consecutive nucleotides of SEQ ID NO: 89 and/or SEQ ID NO: 90 and an antisense region comprising a second sequence complementary to said sense region is disclosed herein. In one embodiment, the dsRNA sense region has at least 99% or has 100% sequence identity to a portion of at least 19 consecutive nucleotides of one or more endogenous HPPR gene(s), such as SEQ ID NO: 89 and/or SEQ ID NO: 90. In some embodiments, the dsRNA of the invention is expressed in a plant cell.

Also disclosed herein is a DNA comprising a promoter functional in a host cell, and a DNA encoding a dsRNA comprising a first and a second region, wherein said first region comprises a sequence with at least 94% sequence identity to a portion of at least 19 consecutive nucleotides of one or more endogenous HPPR genes and wherein said second region is complementary to said first region. In some embodiments, the HPPR gene is a sequence selected from the group consisting of: the RNA form of SEQ ID NO: 89 and SEQ ID NO: 90. In one embodiment of the invention, the host cell is a bacterial cell, a yeast cell, or a plant cell.

Also disclosed herein is a chimeric gene comprising the following operably linked DNA: (a) a plant-expressible promoter; (b) a DNA region which when transcribed yields a double-stranded RNA molecule targeting one or more endogenous HPPR genes of a plant, said RNA molecule comprising a first and second RNA region wherein: (i) said first RNA region comprises a nucleotide sequence of at least 19 consecutive nucleotides having at least 94% sequence identity to the nucleotide sequence of said gene; (ii) said second RNA region comprises a nucleotide sequence complementary to said at least 19 consecutive nucleotides of said first RNA region; and (iii) said first and second RNA region are capable of base-pairing to form a double-stranded RNA molecule between at least said 19 consecutive nucleotides of said first and second region; and (c) optionally, a 3′ end region comprising transcription termination and polyadenylation signals functioning in plant cells. In one embodiment of the invention, the first RNA region comprises a nucleotide sequence of at least 19 consecutive nucleotides having at least 95% sequence identity SEQ ID NO: 89 and/or 90. In another embodiment of the invention, the said first RNA region comprises at least 19 consecutive nucleotides of SEQ ID NO: 89 and/or 90. In yet another embodiment of the invention, between said first and second RNA region, a spacer region containing a plant intron is present. In one embodiment of the invention, the promoter is a constitutive promoter. In yet another embodiment of the invention, a plant cell, plant or seed comprising the chimeric gene or the double-stranded RNA molecule described above is provided.

In a further embodiment, nucleotides encoding herbicide tolerant polypeptides are co-expressed with the dsRNA of the invention. In some embodiments, the herbicide tolerant polypeptide is an HPPD enzyme. In a preferred embodiment, the HPPD enzyme is a mutant HPPD enzyme derived from Pseudomonas fluorescens (Pf-HPPD). In a more preferred embodiment, the HPPD enzyme is an HPPD protein set forth in any of SEQ ID NOs: 3-59 and 78-88, as well as fragments and functional variants thereof.

Also disclosed herein is a method to increase tolerance to HPPD inhibitor herbicides, the method comprising introducing a dsRNA construct that targets one or more endogenous HPPR genes in a plant. In some embodiments, the dsRNA targets the nucleotide sequence of SEQ ID NO: 89 and/or SEQ ID NO: 90. In some embodiments, the method further comprises introducing a nucleotide sequence encoding an herbicide tolerant polypeptide in a plant. In some embodiments, the herbicide tolerant polypeptide encoded by the nucleotide sequence is an HPPD enzyme. In a preferred embodiment, the HPPD enzyme is a mutant HPPD enzyme derived from Pseudomonas fluorescens (Pf-HPPD). In a more preferred embodiment, the HPPD enzyme is an HPPD protein set forth in any of SEQ ID NOs: 3-59 and 78-88, as well as fragments and functional variants thereof.

Transformed plants, plant cells, tissues, and seeds that are tolerant to HPPD inhibitor herbicides by the introduction of the nucleic acids of the invention into the genome of the plants, plant cells, tissues, and seeds are also provided herein.

### BRIEF DESCRIPTION OF THE FIGURES

FIG. 1A-C shows an alignment of amino acid sequence of HPPDs from microbial and plant species, including Pseudomonas fluorescens (SEQ ID NO: 1), Avena sativa (SEQ ID NO:63), a variant of the HPPD from Avena sativa (SEQ ID NO:64), Zea mays (SEQ ID NO:65), Streptomyces avermitilis (SEQ ID NO:69), Arabidopsis thaliana (SEQ ID NO:66), Hordeum vulgare (SEQ ID NO:67), Daucus carota (SEQ ID NO:68), Mycosphaerella graminicola (SEQ ID NO:70), and Coccicoides immitis (SEQ ID NO:71).

FIG. 2 shows an alignment of amino acid sequence of HPPD from Pseudomonas fluorescens (SEQ ID NO: 1) and recombinant Pf-HPPD-evo41 (SEQ ID NO: 16).

FIG. 3 shows the biochemical pathway leading to production of rosmarinic acid catalyzed in part by HPPR.

FIG. 4 shows a hairpin construct designed to silence the expression of the two putative soybean HPPR genes as well as to express HPPD-PFevo41.

FIG. 5 shows an alignment of the concatemer sequence used in the RNAi cassette of pCPE825 (nt 1-307 of SEQ ID NO: 91) and the endogenous putative HPPR gene LOC102662120 (SEQ ID NO: 89).

FIG. 6 shows an alignment of the concatemer sequence used in the RNAi cassette of pCPE825 (nt 308-607 of SEQ ID NO: 91) and the endogenous putative HPPR gene LOC100779623 (SEQ ID NO: 90).

FIG. 7 shows the damage ratings following NOC115 treatment of soybean plants in which the putative HPPR genes are silenced.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### Overview

Several efforts have been developed in order to confer to plants an agronomically-acceptable level of tolerance to a broad range of HPPD inhibitor herbicides, including by-passing HPPD-mediated production of homogentisate (U.S. Pat. No. 6,812,010), overexpressing the sensitive enzyme so as to produce quantities of the target enzyme in the plant which are sufficient in relation to the herbicide (WO96/38567), and mutating the HPPD in order to obtain a target enzyme which, while retaining its properties of catalyzing the transformation of HPP into homogentisate, is less sensitive to HPPD inhibitors than is the native HPPD before mutation.

Despite these successes obtained for the development of plants showing tolerance to several HPPD inhibitors herbicides described above, it is still necessary to develop and/or improve the tolerance of plants to newer or to several different HPPD inhibitors, particularly HPPD inhibitors belonging to the classes of the triketones (e.g. sulcotrione, mesotrione, tembotrione, benzobicyclone and bicyclopyrone), the pyrazolinates (e.g., topramezone and pyrasulfotole), N-(1,2,5-Oxadiazol-3-yl)benzamides (WO 2011/035874), and N-(tetrazol-4-yl)- or N-(triazol-3-yl)arylcarboxamides (WO2012/028579).

Thus, the present invention provides improved compositions and methods for regulating HPPD inhibitor herbicide tolerance. The inventors of the present disclosure surprisingly found that down-regulation of putative 4-hydroxyphenylpyruvate reductases (HPPRs) in soybean resulted in an improvement in herbicide tolerance to HPPD inhibitors.

Terms

As used herein, the singular form “a”, “an” and “the” includes plural references unless the context clearly dictates otherwise. For example, the term “a cell” includes a plurality of cells, including mixtures thereof.

As used herein, the term “gene” refers to a DNA sequence involved in producing a RNA or polypeptide or precursor thereof. The polypeptide or RNA can be encoded by a full-length coding sequence or by intron-interrupted portions of the coding sequence, such as exon sequences. In one embodiment of the invention, the gene target is a HPPR gene.

As used herein, the term “oligonucleotide” refers to a molecule comprising a plurality of deoxyribonucleotides or ribonucleotides. Oligonucleotides may be generated in any manner, including chemical synthesis, DNA replication, reverse transcription, polymerase chain reaction, or a combination thereof. In one embodiment, the present invention embodies utilizing the oligonucleotide in the form of dsRNA as means of interfering with the expression of one or more HPPR enzymes. Inasmuch as mononucleotides are synthesized to construct oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide.

As used herein, the term “primer” refers to an oligonucleotide, which is capable of acting as a point of initiation of synthesis when placed under conditions in which primer extension is initiated. An oligonucleotide “primer” may occur naturally, as in a purified restriction digest or may be produced synthetically.

A primer is selected to be “substantially complementary” to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence is sufficiently complementary with the sequence of the template to hybridize and thereby form a template primer complex for synthesis of the extension product of the primer.

As used herein, “dsRNA” refers to double-stranded RNA that comprises a sense and an antisense portion of a selected target gene (or sequences with high sequence identity thereto so that gene silencing can occur), as well as any smaller double-stranded RNAs formed therefrom by RNAse or dicer activity. Such dsRNA can include portions of single stranded RNA, but contains at least 19 nucleotides double-stranded RNA. In one embodiment of the invention, the dsRNA is a hairpin RNA which contains a loop or spacer sequence between the sense and antisense sequences of the gene targeted, preferably such hairpin RNA spacer region contains an intron.

As used herein, the term “gene silencing” refers to lack of (or reduction of) gene expression as a result of, though not limited to, effects at a genomic (DNA) level such as chromatin re-structuring, or at the post-transcriptional level through effects on transcript stability or translation. Evidence suggests that RNA interference (RNAi) is a major process involved in transcriptional and posttranscriptional gene silencing. Because RNAi exerts its effects at the transcriptional and/or post-transcriptional level, it is believed that RNAi can be used to specifically inhibit alternative transcripts from the same gene.

As used herein, the terms “interfering with” or “inhibiting” (expression of a target sequence) refers to the ability of a small RNA, such as an siRNA or a miRNA, or other molecule, to measurably reduce the expression and/or stability of molecules carrying the target sequence. A target sequence can include a DNA sequence, such as a gene or the promoter region of a gene, or an RNA sequence, such as an mRNA. “Interfering with” or “inhibiting” expression contemplates reduction of the end-product of the gene or sequence, e.g., the expression or function of the encoded protein or a protein, nucleic acid, other biomolecule, or biological function influenced by the target sequence, and thus includes reduction in the amount or longevity of the mRNA transcript or other target sequence. In some embodiments, the small RNA or other molecule guides chromatin modifications which inhibit the expression of a target sequence. It is understood that the phrase is relative, and does not require absolute inhibition (suppression) of the sequence. Thus, in certain embodiments, interfering with or inhibiting expression of a target sequence requires that, following application of the small RNA or other molecule (such as a vector or other construct encoding one or more small RNAs), the sequence is expressed at least 5% less than prior to application, at least 10% less, at least 15% less, at least 20% less, at least 25% less, or even more reduced. Thus, in some particular embodiments, application of a small RNA or other molecule reduces expression of the target sequence by about 30%, about 40%, about 50%, about 60%, or more. In specific examples, where the small RNA or other molecule is particularly effective, expression is reduced by 70%, 80%, 85%, 90%, 95%, or even more.

As used herein, the term “RNA interference” (RNAi) refers to gene silencing mechanisms that involve small RNAs (including miRNA and siRNA) are frequently referred to under the broad term RNAi. Natural functions of RNAi include protection of the genome against invasion by mobile genetic elements such as transposons and viruses, and regulation of gene expression.

RNA interference results in the inactivation or suppression of expression of a gene within an organism. RNAi can be triggered by one of two general routes. First, it can be triggered by direct cellular delivery of short-interfering RNAs (siRNAs, usually about 21 nucleotides in length and delivered in a dsRNA duplex form with two unpaired nucleotides at each 3′ end), which have sequence complementarity to a RNA that is the target for suppression. Second, RNAi can be triggered by one of several methods in which siRNAs are formed in vivo from various types of designed, expressed genes. These genes typically express RNA molecules that form intra- or inter-molecular duplexes (dsRNA) or a “hairpin” configuration which are processed by natural enzymes (DICER or DCL) to form siRNAs. In some cases, these genes express “hairpin”-forming RNA transcripts with perfect or near-perfect base-pairing; some of the imperfect hairpin-forming transcripts yield a special type of small RNA, termed microRNA (miRNA). In either general method, it is the siRNAs (or miRNAs) that function as “guide sequences” to direct an RNA-degrading enzyme (termed RISC) to cleave or silence the target RNA. In some cases, it is beneficial to integrate an RNAi-inducing gene into the genome of a transgenic organism. An example would be a plant that is modified to suppress a specific gene by an RNAi-inducing transgene. In most methods that are currently in practice, RNAi is triggered in transgenic plants by transgenes that express a dsRNA (either intramolecular or hairpin, or intermolecular in which two transcripts anneal to form dsRNA).

As used herein, the term “RNA silencing” is a general term that is used to indicate RNA-based gene silencing or RNAi.

As used herein, the term “silencing agent” or “silencing molecule”, refers to a specific molecule, which can exert an influence on a cell in a sequence-specific manner to reduce or silence the expression or function of a target, such as a target gene or protein. Examples of silencing agents include nucleic acid molecules such as naturally occurring or synthetically generated small interfering RNAs (siRNAs), naturally occurring or synthetically generated microRNAs (miRNAs), naturally occurring or synthetically generated dsRNAs, and antisense sequences (including antisense oligonucleotides, hairpin structures, and antisense expression vectors), as well as constructs that code for any one of such molecules.

As used herein, the term “small interfering RNA” (siRNA) refers to a RNA of approximately 21-25 nucleotides that is processed from a dsRNA by a DICER enzyme (in animals) or a DCL enzyme (in plants). The initial DICER or DCL products are double-stranded, in which the two strands are typically 21-25 nucleotides in length and contain two unpaired bases at each 3′ end. The individual strands within the double stranded siRNA structure are separated, and typically one of the siRNAs then are associated with a multi-subunit complex, the RNAi-induced silencing complex (RISC). A typical function of the siRNA is to guide RISC to the target based on base-pair complementarity.

The term “chimeric” when referring to a gene or DNA sequence is used to refer to a gene or DNA sequence comprising at least two functionally relevant DNA fragments (such as promoter, 5′UTR, coding region, 3′UTR, intron) that are not naturally associated with each other, such as a fusion of functionally relevant DNA fragments from different sources to form a plant-expressible chimeric gene expressing a dsRNA targeting a HPPR gene.

Sequences or parts of sequences which have “high sequence identity”, as used herein, refers to the number of positions with identical nucleotides divided by the number of nucleotides in the shorter of the sequences, being higher than 95%, higher than 96%, higher than 97%, higher than 98%, higher than 99%, or between 96% and 100%. A target gene, or at least a part thereof, as used herein, preferably has high sequence identity to the dsRNA of the invention in order for efficient gene silencing to take place in the target pest. Identity in sequence of the dsRNA or siRNA with a part of the target gene RNA is included in the current invention but is not necessary.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch 1970). A computer-assisted sequence alignment can be conveniently performed using a standard software program such as GAP which is part of the Wisconsin Package Version 10.1 (Genetics Computer Group, Madison, Wis., USA) using the default scoring matrix with a gap creation penalty of 50 and a gap extension penalty of 3.

For the purpose of the invention, the “complement of a nucleotide sequence X” is the nucleotide sequence which would be capable of forming a double-stranded DNA molecule with the represented nucleotide sequence, and which can be derived from the represented nucleotide sequence by replacing the nucleotides by their complementary nucleotide according to Chargaff s rules (A< >T; G< >C) and reading in the 5′ to 3′ direction, i.e., in opposite direction of the represented nucleotide sequence.

In one embodiment of the invention, sense and antisense RNAs can be separately expressed in vitro or in host cells, e.g., from different chimeric gene constructs using the same or a different promoter or from a construct containing two convergent promoters in opposite orientation. These sense and antisense RNAs which are formed, e.g., in the same host cells, can then combine to form dsRNA. It is clear that whenever reference is made herein to a dsRNA chimeric gene or a dsRNA molecule, that such dsRNA formed, e.g., in plant cells, from sense and antisense RNA produced separately is also included. Also synthetically made dsRNA annealing RNA strands are included herein when the sense and antisense strands are present together.

As used herein, the term “concatemer” refers to multiple copies of a DNA sequence arranged end to end in tandem. In particular, two or gene targets may be linked end to end as a concatemer in order to silence each gene target.

A dsRNA “targeting” a HPPR gene, as used herein, refers to a dsRNA that is designed to be identical to or have high sequence identity to an endogenous HPPR gene in plants (the target gene), and as such is designed to silence such gene upon introduction to such plant. One dsRNA can target one or more homologous HPPR target genes in one plant or several homologous HPPR target genes in different plants. In one embodiment, the dsRNA of the invention targets multiple HPPR genes in soybean plants.

The dsRNA chimeric gene, encoding a dsRNA targeting a HPPR gene, can be stably inserted in a conventional manner into the genome of a single plant cell, and the so-transformed plant cell can be used in a conventional manner to produce a transformed plant that has increased resistance to HPPD inhibitor herbicides. For example, a disarmed Ti-plasmid, containing the dsRNA chimeric gene can be used to transform the plant cell, and thereafter, a transformed plant can be regenerated from the transformed plant cell using procedures known in the art. Other types of vectors can be used to transform the plant cell, using procedures such as direct gene transfer, pollen mediated transformation, plant RNA virus-mediated transformation, liposome-mediated transformation, and other methods such as the methods for transforming certain lines of corn (e.g., U.S. Pat. No. 6,140,553; Fromm et al., 1990, Bio/Technology 8, 833-839); Gordon-Kamm et al., 1990, The Plant Cell 2, 603-618) and rice (Shimamoto et al., 1989, Nature 338, 274-276; Datta et al., 1990, Bio/Technology 8, 736-740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (1988, Bio/Technology 6, 915) and Christou et al. (1990, Trends Biotechnology 8, 145) or the method of WO 00/42207.

The resulting transformed plant can be used in a conventional plant breeding scheme to produce more transformed plants with the same characteristics or to introduce the dsRNA chimeric gene in other varieties of the same or related plant species. Seeds, which are obtained from the transformed plants, contain the dsRNA gene as a stable genomic insert. Plants comprising a dsRNA in accordance with the invention include plants comprising or derived from root stocks of plants comprising the dsRNA chimeric gene of the invention, e.g., fruit trees or ornamental plants. Hence, any non-transgenic grafted plant parts inserted on a transformed plant or plant part are included in the invention since the RNA interference signal is transported to these grafted parts and any insects feeding on such grafted plant are similarly affected by the dsRNA or siRNA of the invention.

HPPD inhibitor herbicides of the present disclosure like those of the class of N (1,2,5-oxadiazol-3-yl)benzamides, N-(tetrazol-4-yl)- or N-(triazol-3-yl)arylcarboxamides, such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide, triketones, such as tembotrione, sulcotrione and mesotrione, the class of isoxazoles such as isoxaflutole, or of the class of pyrazolinates, such as pyrasulfotole and topramezone, particularly selected from tembotrione, sulcotrione, topramezone, bicyclopyrone, tefuryltrione, isoxaflutole, and mesotrione, have an outstanding herbicidal activity against a broad spectrum of economically important monocotyledonous and dicotyledonous annual harmful plants. The active substances also act efficiently on perennial harmful plants which produce shoots from rhizomes, wood stocks or other perennial organs and which are difficult to control. Within the meaning of the present invention, “herbicide” is understood as being a herbicidally active substance on its own or such a substance which is combined with an additive which alters its efficacy, such as, for example, an agent which increases its activity (a synergistic agent) or which limits its activity (a safener). The herbicide may further comprise solid or liquid adjuvants or carriers that are ordinarily employed in formulation technology (e.g. natural or regenerated mineral substances, solvents, dispersants, wetting agents, tackifiers, emulsifiers, growth promoting agents, and the like), as well as one or more additional herbicides and/or one or more pesticides (e.g., insecticides, virucides, microbicides, amoebicides, pesticides, fungicides, bacteriocides, nematocides, molluscicides, and the like).

The methods involve transforming organisms with nucleotide sequences encoding an HPPD inhibitor tolerance gene of the invention or otherwise introducing such HPPD inhibitor tolerance genes in organisms not containing them (e.g., by mating, cell fusion, or by crossing organisms containing an introduced HPPD inhibitor gene of the invention with organisms not containing it and obtaining progeny containing such gene). The nucleotide sequences of the invention are useful for preparing plants that show increased tolerance to HPPD inhibitor herbicides, particularly increased tolerance to HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd.2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), and 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone. The HPPD inhibitor herbicide tolerance gene of the invention may also show tolerance towards the “coumarone-derivative herbicides” (described in WO2009/090401, WO2009/090402, WO2008/071918, WO2008/009908). In this regard, any one of the HPPD inhibitor herbicide tolerance genes of the invention can also be expressed in a plant also expressing a chimeric homogentisate solanesyltransferase (HST) gene or a mutated HST gene as described in WO2011/145015, WO2013/064987, WO2013/064964, or WO2010/029311, to obtain plants tolerant to HST inhibitor herbicides. As used herein, a “coumarone-derivative herbicide” or “HST inhibitor herbicide” encompasses compounds which fall under the IUPAC nomenclature of 5H-thiopyrano[4,3-b]pyridin-8-ol, 5H-thiopyrano[3,4-b]pyrazin-8-ol, oxathiino[5,6-b]pyridin-4-ol, and oxathiino[5,6-b]pyrazin-4-ol.

Thus, by “HPPD inhibitor herbicide tolerance” gene of the invention is intended a gene encoding a protein that confers upon a cell or organism the ability to tolerate a higher concentration of an HPPD inhibitor herbicide than such cell or organism that does not express the protein, or to tolerate a certain concentration of an HPPD inhibitor herbicide for a longer time than such cell or organism that does not express the protein, or that confers upon a cell or organism the ability to perform photosynthesis, grow, and/or reproduce with less damage or growth inhibition observed than such cell or organism not expressing such protein. In various embodiments, the HPPD gene of the invention is selected from SEQ ID NOs: 60-62. An “HPPD inhibitor tolerance protein” includes a protein that confers upon a cell or organism the ability to tolerate a higher concentration of HPPD inhibitor herbicide than such cell or organism that does not express the protein, or to tolerate a certain concentration of HPPD inhibitor herbicide for a longer period of time than such cell or organism that does not express the protein, or that confers upon a cell or organism the ability to perform photosynthesis, grow, and/or reproduce with less damage or growth inhibition observed than such cell or organism not expressing such protein. By “tolerate” or “tolerance” is intended either to survive a particular HPPD inhibitor herbicide application, or the ability to carry out essential cellular functions such as photosynthesis, protein synthesis or respiration and reproduction in a manner that is not readily discernable from untreated cells or organisms, or the ability to have no significant difference in yield or even improved yield for plants treated with HPPD inhibitor herbicide compared to such plants not treated with such herbicide (but where weeds have been removed or prevented by a mechanism other than application of the HPPD inhibitor herbicide, such as the methods described in WO2011/100302, which is herein incorporated by reference in its entirety).

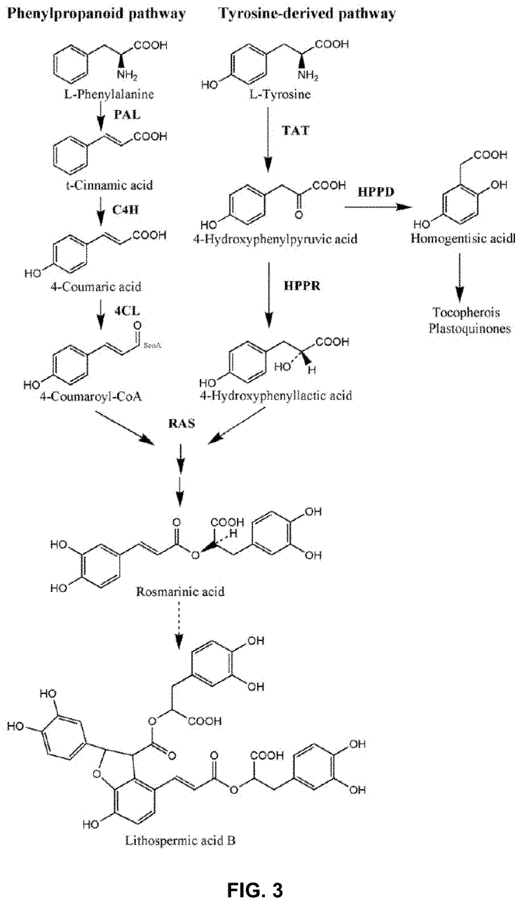

HPPR transforms 4-hydroxyphenylpyruvate to 4-hydroxyphenyllactate, which is then converted to rosmarinic acid by RA synthase as shown in FIG. 3. Thus, HPPR competes for the same substrate as HPPD, namely 4-hydroxyphenylpyruvate. However, it is unknown if this pathway exists in most plant species and it has only been characterized in a few species. According to the instant invention, HPPD inhibitor herbicide tolerance is enhanced by silencing HPPR expression. It is theorized that HPPR silencing results in increased substrate availability for HPPD, leading to increased tolerance to HPPD inhibitors.

The HPPRs of the invention can be any endogenous HPPR protein. For the purposes of describing the HPPRs of the present invention, the terms “protein” and “polypeptide” and “enzyme” are used interchangeably.

In some embodiments, the HPPR protein is a soybean HPPR, such as the HPPR proteins set forth herein as SEQ ID NO: 92 and 93.

In some embodiments, HPPD inhibitor tolerance is further enhanced by expressing at least one HPPD nucleic acid sequence encoding a polypeptide having HPPD activity i.e., catalyzing the reaction in which para-hydroxyphenylpyruvate (HPP) is transformed into homogentisate. The catalytic activity of an HPPD enzyme may be defined by various methods well-known in the art. WO2009/144079 describes various suitable screening methods.

For the purposes of the present invention, a “reference” HPPD protein (or HPPD gene) is any HPPD protein or nucleic acid against which the HPPD protein or HPPD nucleic acid of the invention is being compared. For the purposes of describing the HPPD proteins of the present invention, the terms “protein” and “polypeptide” are used interchangeably. This reference HPPD can be a native plant, bacterial, or animal HPPD, or can be a mutated HPPD that is known in the art such as the PfP215L and PfG336W mutants described in International Patent Publication WO2009/144079 or can be either of the PfHPPDevo33, PfHPPDevo36, PfHPPDevo37, PfHPPDevo40, or PfHPPDevo41, Axmi309H, Axmi428H, Axmi309H-Evo41, or Axmi428H-Evo41 proteins set forth herein as SEQ ID NO:6, 7, 3, 8, 16, 58, 59, 54, and 56, respectively, which are also described in WO2014/043435, which is herein incorporated by reference. Such reference HPPD can be used to determine whether the HPPD protein or nucleic acid of the invention has a particular property of interest (e.g., improved, comparable or decreased HPPD inhibitor herbicide tolerance or HPPD enzyme activity; improved, comparable or decreased expression in a host cell; improved, comparable or decreased protein stability, and the like).

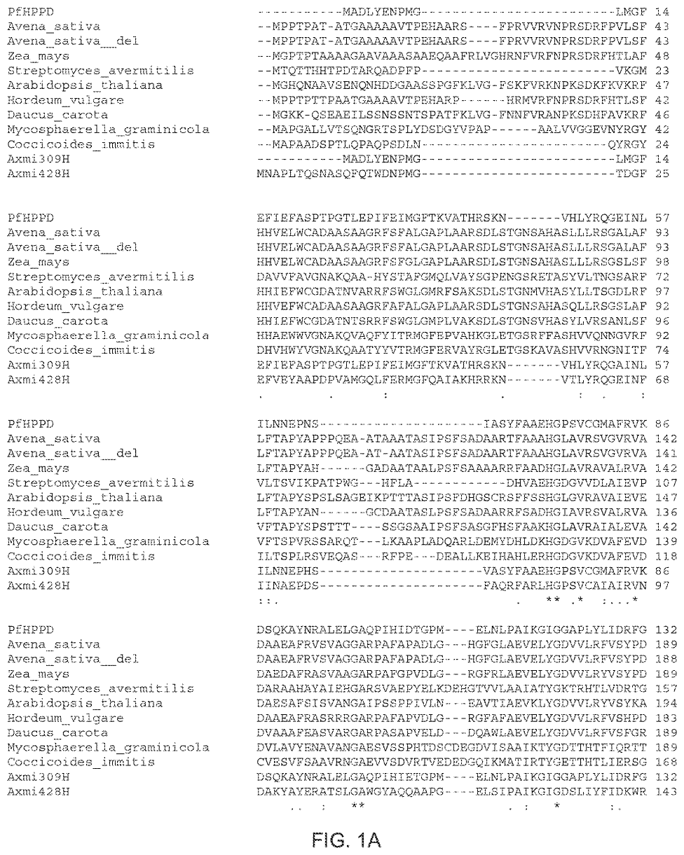

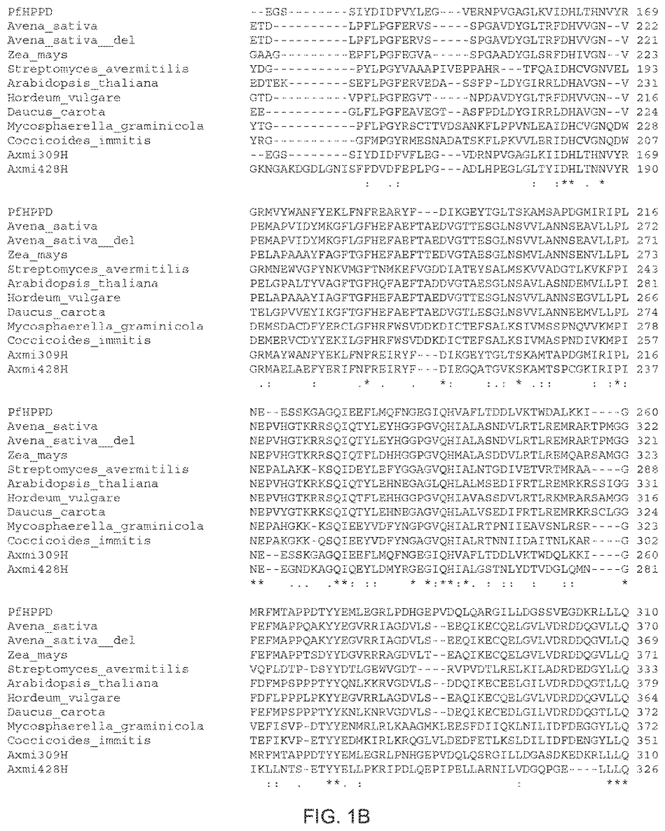

In various embodiments herein, the HPPD inhibitor herbicide tolerant protein encoded by a nucleic acid (including isolated, recombinant and chimeric genes thereof, vectors, host cells, plants, plant parts, and seeds comprising the nucleic acid, HPPD polypeptides and compositions thereof encoded by the nucleic acid, as well as methods of using the protein encoded by the nucleic acid for increasing tolerance of a plant to HPPD inhibitor herbicides, particularly increased tolerance to HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd.2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), and 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone) has been modified to contain one or more amino acid substitutions, including 2, 3, 4, 5, 6, or 7 amino acid substitutions, at the positions corresponding to amino acid positions 188, 189, 215, 335, 336, 339, and/or 340 of SEQ ID NO:1. By “corresponding to” is intended the nucleotide or amino acid position relative to that position in SEQ ID NO:1 when two (or more) sequences are aligned using standard alignment algorithms described elsewhere herein. A representative alignment of SEQ ID NO:1 with HPPD amino acid sequences from various microbial and plant species is shown in FIG. 1A-C. For example, amino acid positions 188, 189, 215, 335, 336, 339, and 340 of SEQ ID NO:1 correspond to amino acid positions 241, 242, 271, 412, 413, 416, and 417, respectively, of the HPPD from Avena sativa (SEQ ID NO:63); to amino acid positions 235, 236, 265, 406, 407, 410, and 411, respectively, of the HPPD from Hordeum vulgare (SEQ ID NO:67) and to amino acid positions 242, 243, 272, 413, 414, 417, and 418, respectively, of the HPPD from Zea mays (SEQ ID NO:65). Accordingly, depending on the length of the concerned HPPD amino acid sequence, having either additional or fewer residues than the sequence of SEQ ID NO:1, the corresponding position can be located at a position different from positions 188, 189, 215, 335, 336, 339, and 340 in such concerned HPPD protein.

In one embodiment, the HPPD of the present invention has been modified to comprise one or more amino acid substitution(s) selected from the group consisting of:

(a) a tryptophan, glycine, or serine at the amino acid position corresponding to amino acid position 188 of SEQ ID NO:1;

(b) a serine, cysteine, or arginine at the amino acid position corresponding to amino acid position 189 of SEQ ID NO:1;

(c) a proline, serine, histidine, alanine, glycine, or glutamine at the amino acid position corresponding to amino acid position 335 of SEQ ID NO:1;

(d) a serine or tryptophan at the amino acid position corresponding to amino acid position 336 of SEQ ID NO:1;

(e) a threonine, alanine, or serine at the amino acid position corresponding to amino acid position 339 of SEQ ID NO:1;

(f) a glutamine, alanine, valine, or glutamic acid at the amino acid position corresponding to amino acid position 340 of SEQ ID NO:1; and

(g) a leucine at the amino acid position corresponding to amino acid position 215 of SEQ ID NO:1.

In another embodiment, the HPPD has been modified to comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In specific embodiments, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence set forth herein as SEQ ID NO:1, wherein the HPPD has been modified to comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence set forth herein as SEQ ID NO:1 and wherein said HPPD comprises the amino acid substitution(s) of:

(a) a tryptophan at amino acid position 188 and a tryptophan at amino acid position 336; or

(b) a proline at amino acid position 335.

In yet another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence set forth herein as SEQ ID NO:63, wherein the HPPD has been modified to comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1 and a serine at the amino acid position corresponding to amino acid position 336 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In yet another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence set forth herein as SEQ ID NO:64, wherein the HPPD has been modified to comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1 and a serine at the amino acid position corresponding to amino acid position 336 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In yet another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence set forth herein as SEQ ID NO:65, wherein the HPPD has been modified to comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, and a threonine at the amino acid position corresponding to amino acid position 339 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, and an alanine at the amino acid position corresponding to amino acid position 339 of SEQ ID NO:1; or

(f) a proline at the amino acid position corresponding to amino acid position 335 of SEQ ID NO:1.

In another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth herein as SEQ ID NO:57, or is encoded by a nucleotide sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the nucleotide sequence set forth herein as SEQ ID NO:60. The HPPD of this embodiment may further comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1; an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth herein as SEQ ID NO:58, or is encoded by a nucleotide sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the nucleotide sequence set forth herein as SEQ ID NO:61. The HPPD of this embodiment may further comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1; an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 53%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth herein as SEQ ID NO:59, or is encoded by a nucleotide sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the nucleotide sequence set forth herein as SEQ ID NO:62. The HPPD of this embodiment may further comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1; an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 85% sequence identity to the amino acid sequence set forth herein as SEQ ID NO:1, wherein the HPPD has been modified to comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 85% sequence identity to the amino acid sequence set forth herein as SEQ ID NO:1 and wherein said HPPD comprises the amino acid substitution(s) of:

(a) a tryptophan at amino acid position 188 and a tryptophan at amino acid position 336; or

(b) a proline at amino acid position 335.

In another embodiment, the HPPD of the invention has at least 85% sequence identity to the amino acid sequence set forth herein as SEQ ID NO:57. The HPPD of this embodiment may further comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 85% sequence identity to the amino acid sequence set forth herein as SEQ ID NO:58. The HPPD of this embodiment may further comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

In another embodiment, the HPPD of the invention has at least 85% sequence identity to the amino acid sequence set forth herein as SEQ ID NO:59. The HPPD of this embodiment may further comprise amino acid substitution(s) of:

(a) a proline at the amino acid position corresponding to amino acid position 335 of SEQ ID NO:1 and a tryptophan at the amino acid position corresponding to amino acid position 336 of SEQ ID NO:1;

(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;

(c) a tryptophan at the amino acid position corresponding to amino acid position 188 of SEQ ID NO:1 and a tryptophan at the amino acid position corresponding to amino acid position 336 of SEQ ID NO:1;

(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1;

(e) a proline at the amino acid position corresponding to amino acid position 335 of SEQ ID NO:1, a tryptophan at the amino acid position corresponding to amino acid position 336 of SEQ ID NO:1; an alanine at the amino acid position corresponding to amino acid position 339 of SEQ ID NO:1, and a glutamine at the amino acid position corresponding to amino acid position 340 of SEQ ID NO:1; or

(f) a proline at the amino acid position corresponding to amino acid position 335 of SEQ ID NO:1.

Any HPPD sequence can be modified to contain one or more of the substitutions disclosed herein. For example, the HPPD of the invention also encompasses any naturally-occurring bacterial, plant, or animal HPPD enzymes that has been modified to contain one or more of the substitutions described supra.

In arriving at the HPPD protein of the current invention, a starting amino acid sequence of an existing protein has to be modified by man by replacing at least one amino acid as defined in the present application, which is most conveniently done by modifying the DNA encoding such protein by replacing a certain codon by another codon encoding another amino acid.

Exemplary HPPD sequences that can be modified according to the present invention include those from bacteria, for example, of the Pseudomonas sp. type, for example Pseudomonas fluorescens, or otherwise cyanobacteria of the Synechocystis genus. The sequence can also be of plant origin, in particular derived from dicotyledonous plants, umbelliferous plants, or otherwise monocotyledonous plants. Advantageous examples which may be cited are plants such as tobacco, Arabidopsis, Daucus carotta, Zea mays (corn), wheat, barley, Avena sativa, Brachiaria platyphylla, Cenchrus echinatus, Lolium rigidum, Festuca arundinacea, Setaria faberi, Eleusine indica, Sorghum. The coding sequences, and the way of isolating and cloning them, are known in the art or described elsewhere herein (e.g., SEQ ID NO:63-76). In a particular embodiment of the invention, the HPPD that can be modified according to the present invention is from a bacterial origin, particularly from Pseudomonas sp., more particularly from Pseudomonas fluorescens, Rhodococcus sp Blepharisma japonicum, Synechococcus sp., Picrophilus torridus, Kordia algicida or from a plant origin, including from Arabidopsis thaliana, Sorghum bicolor, Oryza sativa, Triticum aestivum, Hordeum vulgare, Lolium rigidum, or Avena sativa.

For the purposes of the present invention, the HPPD of the invention may also comprise further modifications, for example, wherein some amino acids (e.g., 1 to 10 amino acids) have been replaced, added or deleted for cloning purposes, to make a transit peptide fusion, and the like, which retains HPPD activity, i.e. the property of catalyzing the conversion of para-hydroxyphenylpyruvate to homogentisate, or can be any HPPD that can be further improved. For example, the HPPD that can be further improved by the modifications described herein can be the variant HPPD derived from Pseudomonas fluorescens set forth herein as SEQ ID NO:2, the variant HPPD from Avena sativa set forth herein as SEQ ID NO:64, the variant HPPD sequences set forth in any of SEQ ID NO:3-326, 383-389, 393, 395, and 397-459 in WO2012/021785, which is herein incorporated by reference in its entirety; the HPPD sequences set forth in any of SEQ ID NO:2-14 and 20-50 of WO2011/068567, which is herein incorporated by reference in its entirety; the HPPD sequences set forth in any of SEQ ID NO:15-26 of WO2010/085705, which is herein incorporated by reference in its entirety; an HPPD having one or more of the substitutions described in WO09/144079 or U.S. Pat. No. 6,245,968, each of which is herein incorporated by reference in its entirety; an HPPD having one or more of the substitutions described in Tables 1, 2, 5, or 6 of WO2010/085705; and/or an HPPD having one or more of the substitutions described in Table 1 of WO2011/068567; the variant HPPD sequences set forth in any of SEQ ID NO:3-59 of WO2014/043435; or an HPPD having one or more of the substitutions described in Table 1 of WO2015/0138394, which is herein incorporated by reference in its entirety.

In some embodiments, the nucleotide sequence of the invention (including isolated, recombinant and chimeric genes thereof, vectors, host cells, plants, plant parts, and seeds comprising the nucleic acid sequence, amino acid sequences and compositions thereof encoded by the nucleic acid sequence, as well as methods of using the nucleic acid sequence for increasing tolerance of a plant to HPPD inhibitor herbicides, particularly increased tolerance to HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4)t 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone) encodes the amino acid sequence set forth in any one of SEQ ID NO:3-59 and 78-88, and fragments and variants thereof that encode a HPPD inhibitor herbicide tolerance polypeptide. Thus, in this embodiment, the HPPD of the invention comprises the amino acid sequence set forth in any of SEQ ID NO:3-59 and 78-88, and fragments and variants thereof, that confer tolerance to HPPD inhibitor herbicides in a host cell.

A. Methods for Measuring HPPD Inhibitor Tolerance

Any suitable method for measuring tolerance to HPPD inhibitor herbicides can be used to evaluate the transformants of the invention. Tolerance can be measured by monitoring the ability of a cell or organism to survive a particular HPPD inhibitor herbicide application, or the ability to carry out essential cellular functions such as photosynthesis, protein synthesis or respiration and reproduction in a manner that is not readily discernable from untreated cells or organisms, or the ability to have no significant difference in yield or even improved yield for plants treated with HPPD inhibitor herbicide compared to such plants not treated with such herbicide (but where weeds have been removed or prevented by a mechanism other than application of the HPPD inhibitor herbicide). In some embodiments, tolerance can be measured according to a visible indicator phenotype of the cell or organism transformed with a nucleic acid comprising the RNAi region designed to silence the endogenous HPPR protein(s) and/or the gene coding for the respective HPPD protein, or in an in vitro assay of the HPPD protein, in the presence of different concentrations of the various HPPD inhibitors. Dose responses and relative shifts in dose responses associated with these indicator phenotypes (formation of brown color, growth inhibition, bleaching, herbicidal effect etc.) are conveniently expressed in terms, for example, of GR50 (concentration for 50% reduction of growth) or MIC (minimum inhibitory concentration) values where increases in values correspond to increases in inherent tolerance to HPPD inhibitors, in the normal manner based upon plant damage, meristematic bleaching symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent tolerance to HPPD inhibitors. Herbicides can suitably be applied pre-emergence or post emergence.

In various embodiments, tolerance level of the transformants of the invention can be screened via transgenesis, regeneration, breeding and spray testing of a test plant such as tobacco, or a crop plant such as soybean, corn, or cotton. In line with the results obtained by such screening, such plants are more tolerant, desirably tolerant to at least 2 times the normal dose recommended for field applications, even more preferably tolerant up to 4 times the normal dose recommended for field applications, to HPPD inhibitor herbicides (e.g., HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone) than such plants that express normal levels of HPPR enzymes and/or do not contain any exogenous gene encoding an HPPD protein, or than plants that contain a gene comprising a reference HPPD-encoding DNA, for example, a Pseudomonas fluorescens HPPD-encoding DNA, under control of the same promoter as the nucleic acid encoding the HPPD protein of the invention. Accordingly, the term “capable of increasing the tolerance of a plant to at least one herbicide acting on HPPD” denotes a tolerance by the plant expressing the HPPD of the invention to at least 1×, 2×, or 3×, or 4×, or greater, the normal field dose of the HPPD inhibitor herbicide as compared to a plant only expressing its endogenous HPPD or a plant expressing a reference HPPD enzyme. In this regard, the term “herbicide acting on HPPD” is not limited to substances which are known and/or used as herbicides but to any substances which inhibit the catalytic activity of HPPD proteins.

Alternatively, at the quantitative level, data like pI₅₀ (pI₅₀-value means the log value of the concentration of inhibitor necessary to inhibit 50% of the enzyme activity in molar concentration) can be obtained for the transformants of the invention and compared to a cell expressing normal levels of HPPR enzyme(s) and/or a reference HPPD sequence in the presence or absence of any respective HPPD inhibitor herbicide.

A specific, although non-limiting, type of assay that can be used to evaluate the transformants of the invention is a colorimetric assay. In this assay, a YT-broth-type culture medium with 1% agarose, 5 mM L-Tyrosine and 42 mM Succinate, which contains the selection agent for the vector pSE420 (Invitrogen, Karlsruhe, Germany) or a modified version of pSE420 (pSE420(RI)NX) is poured into deep well plates. E. coli culture in the exponential growth phase which contains the vector pSE420-HPPDx (HPPDx means any gene coding for a putative HPPD enzyme/protein) is applied to each well. After 16 hours at 37° C., the wells which do not contain the culture medium, those which have been seeded with an E. coli culture containing the empty vector pSE420 are transparent, or those which have been seeded with an E. coli culture containing a vector pSE420-HPPDx containing a gene coding for an inactive HPPD are transparent, while the wells seeded with an E. coli culture containing the vector pSE420-HPPDx coding for an active HPPD are brown. It has been previously demonstrated that this test reflects the HPPD activity, whatever the origin of this activity is, and allows the identification of HPPD activities (U.S. Pat. No. 6,768,044), i.e. at a qualitative level.

B. Methods of Reducing HPPR Expression in Plant Cells

In a preferred embodiment of the invention, HPPR expression is reduced using RNA interference as described above. Accordingly, one or more genes encoding HPPR may be targeted.

Information on how to design optimal dsRNA sequences once a target gene is known can be found with commercial providers, e.g., the companies Ambion and Cenix BioScience (Ambion Inc., 2130 Woodward Street, Austin, Tex. 78744-1832, USA; and see www.ambion.com; and Cenix BioScience GmbH, Pfotenhauerstr. 108, 01307 Dresden, Germany, see www.cenix-bioscience.com). Preferably, the dsRNAs to be used in this invention target at least one HPPR plant gene, or a HPPR plant gene portion of at least 19 consecutive nucleotides occurring in identical sequence or with high sequence identity in a several plant species. In one embodiment, a portion of a target HPPR gene sequence is selected which is present in several plant hosts with identical sequence or with high sequence identity, of a length sufficient to be capable of silencing the HPPR gene.

In one embodiment of this invention, the dsRNA or siRNA of the invention corresponds to an exon in the target gene.

Also, in the dsRNA chimeric gene of the invention a nuclear localization signal can be added as described in published US patent application 20030180945 (incorporated herein by reference).

As used herein, nucleotide sequences of RNA molecules may be identified by reference to DNA nucleotide sequences of the sequence listing. However, the person skilled in the art will understand whether RNA or DNA is meant depending on the context. Furthermore, the nucleotide sequence is identical except that the T-base is replaced by uracil (U) in RNA molecules.

The length of the first (e.g., sense) and second (e.g., antisense) nucleotide sequences of the dsRNA molecules of the invention may vary from about 10 nucleotides (nt) up to a length equaling the length in nucleotides of the transcript of the target gene. The length of the first or second nucleotide sequence of the dsRNA of the invention can be at least 15 nt, or at least about 20 nt, or at least about 50 nt, or at least about 100 nt, or at least about 150 nt, or at least about 200 nt, or at least about 400 nt, or at least about 500 nt. If not all nucleotides in a target gene sequence are known, it is preferred to use such portion for which the sequence is known and which meets other beneficial requirements of the invention.

It will be appreciated that the longer the total length of the first (sense) nucleotide sequence in the dsRNA of the invention is, the less stringent the requirements for sequence identity between the total sense nucleotide sequence and the corresponding sequence in the target gene becomes. The total first nucleotide sequence can have a sequence identity of at least about 75% With the corresponding target sequence, but higher sequence identity can also be used such as at least about 80%, at least about 85%, at least about 90%, at least about 95%, about 100%. The first nucleotide sequence can also be identical to the corresponding part of the target gene. However, it is advised that the first nucleotide sequence includes a sequence of 19 or 20, or about 19 or about 20 consecutive nucleotides, or even of about 50 consecutive nucleotides, or about consecutive 100 nucleotides, or about 150 consecutive nucleotides with only one mismatch, preferably with 100% sequence identity, to the corresponding part of the target gene. For calculating the sequence identity and designing the corresponding first nucleotide sequence, the number of gaps should be minimized, particularly for the shorter sense sequences.

The length of the second (antisense) nucleotide sequence in the dsRNA of the invention is largely determined by the length of the first (sense) nucleotide sequence, and may correspond to the length of the latter sequence. However, it is possible to use an antisense sequence which differs in length by about 10% without any difficulties. Similarly, the nucleotide sequence of the antisense region is largely determined by the nucleotide sequence of the sense region, and may be identical to the complement of the nucleotide sequence of the sense region. Particularly with longer antisense regions, it is possible to use antisense sequences with lower sequence identity to the complement of the sense nucleotide sequence, such as at least about 75% sequence identity, or least about 80%, or at least about 85%, more particularly with at least about 90% sequence identity, or at least about 95% sequence to the complement of the sense nucleotide sequence. Nevertheless, it is advised that the antisense nucleotide sequence always includes a sequence of 19 or 20, about 19 or about 20 consecutive nucleotides, although longer stretches of consecutive nucleotides such as about 50 nucleotides, or about 100 nucleotides, or about 150 nucleotides with no more than one mismatch, preferably with 100% sequence identity, to the complement of a corresponding part of the sense nucleotide sequence can also be used. Again, the number of gaps should be minimized, particularly for the shorter (19 to 50 nucleotides) antisense sequences.

In one embodiment of the invention, the DNA molecules according to the invention may comprise a DNA region encoding a spacer between the DNA region encoding the first and second nucleotide sequences. As indicated in WO 99/53050 the spacer may contain an intron to enhance gene silencing. A particularly preferred intron functional in cells of plants is the pdk intron (Flaveria trinervia pyruvate orthophosphate dikinase intron 2; see WO99/53050 incorporated by reference), the delta 12 desaturase intron from Arabidopsis (Smith et al., 2000, Nature 407:319-20) or the intron of the rolA gene (Magrelli et al., 1994, Science 266: 1986-1988; Spena and Langenkemper, 1997, Genet. Res. 69:11-15).

In one embodiment of the invention, the dsRNA molecule may further comprise one or more regions having at least 94%, at least 95%, at least 96%, at least 97%, at least 98%, or at least 99%, sequence identity to regions of at least 19 consecutive nucleotides from the sense nucleotide sequence of the target gene, different from the at least 19 consecutive nucleotides as defined in the first region, and one or more regions having at least 94%, at least 95%, at least 96%, at least 97%, at least 98%, or at least 99%, sequence identity to at least 19 consecutive nucleotides from the complement of the sense nucleotide sequence of the target gene, different from the at least 19 consecutive nucleotides as defined in the second region, wherein these additional regions can basepair amongst themselves.

“Substantially identical” as used herein, means there is a very high degree of homology (preferably 100% sequence identity) between the inhibitory dsRNA and the corresponding part of the target gene. However, dsRNA having greater than 90% or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base pair random mismatches between the RNA and the target gene.

Methods which are well known to those skilled in the art may be used to construct expression vectors containing sequences encoding HPPR gene and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

While the examples provided herein describe dsRNA constructs cloned from GenBank Accession Nos. NM_001317538 (gene ID: LOC100779623; Glycine max) and XM_014767802 (gene ID: LOC102662120; Glycine max), it is contemplated that when read in conjunction with the teaching disclosed herein and the knowledge in the art, the construction of other dsRNA constructs targeting HPPR gene sequences of other plant species would be feasible to those skilled the in the art. For example, including but not limited to the HPPR gene/amino acid sequences disclosed herein, it is contemplated that a dsRNA construct targeting other plant species would increase tolerance to HPPD inhibitor herbicides in those plants. Additionally, it is contemplated that a single dsRNA construct would be effective in increasing tolerance to HPPD inhibitor herbicides in a plurality of plant species.

C. Methods of Introducing Mutations into HPPD Sequences

In the mutated HPPD protein encoded by the nucleic acid of the invention at least one amino acid has been replaced as defined above.

The replacement can be effected in the nucleic acid sequence which encodes the reference HPPD as defined above by any means which is appropriate for replacing, in the said sequence, the codon which encodes the amino acid to be replaced with the codon which corresponds to the amino acid which is to replace it, with the said codons being widely described in the literature and well known to the skilled person.

Several molecular biological methods can be used to achieve this replacement. A useful method for preparing a mutated nucleic acid sequence according to the invention and the corresponding protein comprises carrying out site-directed mutagenesis on codons encoding one or more amino acids which are selected in advance. The methods for obtaining these site-directed mutations are well known to the skilled person and widely described in the literature (in particular: Directed Mutagenesis: A Practical Approach, 1991, Edited by M. J. McPHERSON, IRL PRESS), or are methods for which it is possible to employ commercial kits (for example the QUIKCHANGE™ lightening mutagenesis kit from Qiagen or Stratagene). After the site-directed mutagenesis, it is useful to select the cells which contain a mutated HPPD which is less sensitive to an HPPD inhibitor by using an appropriate screening aid. Appropriate screening methods to achieve this have been described above.

Alternatively, a DNA sequence encoding the reference HPPD can be modified in silico to encode an HPPD protein having one or more of the substitutions recited herein, and then synthesized de novo. The nucleotide sequence encoding the mutated HPPD protein can be introduced into a host cell as described elsewhere herein.

D. Isolated Polynucleotides, and Variants and Fragments Thereof

In some embodiments, the present invention comprises isolated or recombinant, polynucleotides. A “recombinant” polynucleotide or polypeptide/protein, or biologically active portion thereof, as defined herein is no longer present in its original, native organism, such as when contained in a heterologous host cell or in a transgenic plant cell, seed or plant. In one embodiment, a recombinant polynucleotide is free of sequences (for example, protein encoding or regulatory sequences) that naturally flank the nucleic acid (i.e., sequences located at the 5′ and 3′ ends of the nucleic acid) in the genomic DNA of the organism from which the polynucleotide is derived. The term “recombinant” encompasses polynucleotides or polypeptides that have been manipulated with respect to the native polynucleotide or polypeptide, such that the polynucleotide or polypeptide differs (e.g., in chemical composition or structure) from what is occurring in nature. In another embodiment, a “recombinant” polynucleotide is free of internal sequences (i.e. introns) that naturally occur in the genomic DNA of the organism from which the polynucleotide is derived. A typical example of such polynucleotide is a so-called Complementary DNA (cDNA). For example, in various embodiments, the isolated HPPD inhibitor herbicide tolerance-encoding polynucleotide can contain less than about 5 kb, 4 kb, 3 kb, 2 kb, 1 kb, 0.5 kb, or 0.1 kb of nucleotide sequence that naturally flanks the polynucleotide in genomic DNA of the cell from which the polynucleotide is derived. Nucleic acid molecules of the invention include those designed to silence the HPPR(s) of the invention and/or those that encode the HPPD of the invention. In some embodiments, the nucleic acid molecule of the invention is operably linked to a promoter capable of directing expression of the nucleic acid molecule in a host cell (e.g., a plant host cell or a bacterial host cell).

In some embodiments, the polynucleotides of the invention include fragments of HPPR genes for use in silencing endogenous HPPR genes by repression of transcription as discussed above, such as dsRNA, hairpin RNA, and/or complementary RNA.

The present invention further contemplates variants and fragments of any nucleic acid sequence encoding the amino acid sequences set forth in any of SEQ ID NO:1-59, 78-88, 92, and 93. A “fragment” of a polynucleotide may encode a biologically active portion of a polypeptide, or it may be a fragment that can be used as a hybridization probe or PCR primer using methods disclosed elsewhere herein. Polynucleotides that are fragments of a polynucleotide comprise at least about 15, 20, 50, 75, 100, 200, 300, 350, 400, 450, 500, 550, 600, 650, 700, 750, 800, 850, 900, 950, 1000, 1050, 1100, 1150, contiguous nucleotides, or up to the number of nucleotides present in a full-length polynucleotide disclosed herein depending upon the intended use (e.g., an HPPR or HPPD nucleic acid described herein). By “contiguous” nucleotides are intended nucleotide residues that are immediately adjacent to one another.

Fragments of the HPPD polynucleotides of the present invention generally will encode polypeptide fragments that retain the biological activity of the full-length HPPD inhibitor herbicide tolerance protein; i.e., herbicide-tolerance activity. By “retains herbicide tolerance activity” is intended that the fragment will have at least about 30%, at least about 50%, at least about 70%, at least about 80%, 85%, 90%, 95%, 100%, 110%, 125%, 150%, 175%, 200%, 250%, at least about 300% or greater of the herbicide tolerance activity of the full-length HPPD inhibitor herbicide tolerance protein disclosed herein as SEQ ID NO:3-59 and 78-88. Methods for measuring herbicide tolerance activity are well known in the art and exemplary methods are described herein. In a non-limiting example, a fragment of the invention will be tolerant to the same dose of an HPPD inhibitor herbicide, or tolerant to 1×, 2×, 3×, 4×, or higher dose of an HPPD inhibitor herbicide, or the fragments will be as or more tolerant based on pI50 or Ki between the fragment and SEQ ID NO:3-59 and 78-88.

A fragment of a polynucleotide that encodes a biologically active portion of a polypeptide of the invention will encode at least about 150, 175, 200, 250, 300, 350 contiguous amino acids, or up to the total number of amino acids present in a full-length polypeptide of the invention. In a non-limiting example, a fragment of a polynucleotide that encodes a biologically active portion of a HPPD protein having a proline at the amino acid position corresponding to amino acid position 335 of SEQ ID NO:1 and a phenylalanine or a tyrosine at the position corresponding to amino acid position 336 of SEQ ID NO:1 and, optionally, one or more amino acid substitutions at the positions corresponding to amino acid positions 172, 188, 200, 226, 339, and 340 of SEQ ID NO:1., including the HPPD protein set forth in any of SEQ ID NO:3-59 and 78-88.

The invention also encompasses variant polynucleotides as described supra. “Variants” of the polynucleotide also include those sequences that encode the HPPR and/or HPPD of the invention but that differ conservatively because of the degeneracy of the genetic code, as well as those that are sufficiently identical. In some embodiments, variants of the present invention will retain HPPD enzyme activity and HPPD herbicide inhibitor tolerance. The term “sufficiently identical” is intended a polypeptide or polynucleotide sequence that has at least about 53%, at least about 60% or 65% sequence identity, about 70% or 75% sequence identity, about 80% or 85% sequence identity, about 90%, 91%, 92%, 93%, 94%, 95%, 96%, 97%, 98% or 99% sequence identity compared to a reference sequence using one of the alignment programs using standard parameters. One of skill in the art will recognize that these values can be appropriately adjusted to determine corresponding identity of polypeptides encoded by two polynucleotides by taking into account codon degeneracy, amino acid similarity, reading frame positioning, and the like.

Bacterial genes quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. Furthermore, it is not often determined a priori which of these codons are used naturally in the bacterium. Thus, it is understood that use of one of the alternate methionine codons may lead to generation of variants that confer herbicide tolerance. These herbicide tolerance proteins are encompassed in the present invention and may be used in the methods of the present invention. Naturally occurring allelic variants can be identified with the use of well-known molecular biology techniques, such as polymerase chain reaction (PCR) and hybridization techniques as outlined below. Variant polynucleotides also include synthetically derived polynucleotides that have been generated, for example, by using site-directed or other mutagenesis strategies but which still encode the polypeptide having the desired biological activity.

The skilled artisan will further appreciate that changes can be introduced by further mutation of the polynucleotides of the invention thereby leading to further changes in the amino acid sequence of the encoded polypeptides, without altering the biological activity of the polypeptides. Thus, variant isolated polynucleotides can be created by introducing one or more additional nucleotide substitutions, additions, or deletions into the corresponding polynucleotide encoding the HPPR and/or HPPD of the invention, such that 1-5, 1-10, or 1-15 amino acid substitutions, additions or deletions, or 1, 2, 3, 4, 5, 6, 7, 8, 9, 10, 11, 12, 13, 14, or 15 amino acid substitutions, additions or deletions, are introduced into the encoded polypeptide. Further mutations can be introduced by standard techniques, such as site-directed mutagenesis and PCR-mediated mutagenesis, or gene shuffling techniques. Such variant polynucleotides are also encompassed by the present invention.

Variant polynucleotides can be made by introducing mutations randomly along all or part of the coding sequence, such as by saturation mutagenesis or permutational mutagenesis, and the resultant mutants can be screened for the ability to confer herbicide tolerance activity to identify mutants that retain activity.

Additional methods for generating variants include subjecting a cell expressing a protein disclosed herein (or library thereof) to a specific condition that creates a stress to the activity of the protein. Specific conditions can include (but are not limited to) changes in temperature, changes in pH, and changes in the concentrations of substrates or inhibitors. The protein library can be subjected to these conditions during the time of protein expression (e.g., in E. coli or other host) or following creation of a protein extract, or following protein purification.

The functional or enzymatic activity of the protein library that has been subjected to a stress condition can then be compared to the reference protein to identify proteins with improved properties. This activity comparison can be carried out as part of a growth screen or alternatively as part of an enzymatic assay that quantifies the activity of the protein. The properties that can be identified as improved can include HPPD inhibitor herbicide tolerance, changes in kinetic constants (including Km, Ki, k_{cat}), protein stability, protein thermostability, or protein temperature and pH optimum.

E. Isolated Proteins and Variants and Fragments Thereof

Herbicide tolerance polypeptides are also encompassed within the present invention. An herbicide tolerance polypeptide includes preparations of polypeptides having less than about 30%, 20%, 10%, or 5% (by dry weight) of non-herbicide tolerance polypeptide (also referred to herein as a “contaminating protein”). In the present invention, “herbicide tolerance protein” is intended an HPPD polypeptide disclosed herein. Fragments, biologically active portions, and variants thereof are also provided, and may be used to practice the methods of the present invention.

“Fragments” or “biologically active portions” include polypeptide fragments comprising a portion of an amino acid sequence encoding an herbicide tolerance protein and that retains herbicide tolerance activity. A biologically active portion of an herbicide tolerance protein can be a polypeptide that is, for example, 10, 25, 50, 100 or more amino acids in length. Such biologically active portions can be prepared by recombinant techniques and evaluated for herbicide tolerance activity.

By “variants” is intended proteins or polypeptides having an amino acid sequence that is at least about 53%, 60%, 65%, about 70%, 75%, about 80%, 85%, 90%, 91%, 92%, 93%, 94%, 95%, 96%, 97%, 98% or 99% identical to any of SEQ ID NO:3-59 and 78-88, wherein said variant has HPPD enzyme activity and HPPD inhibitor herbicide tolerance One of skill in the art will recognize that these values can be appropriately adjusted to determine corresponding identity of polypeptides encoded by two polynucleotides by taking into account codon degeneracy, amino acid similarity, reading frame positioning, and the like.

For example, conservative amino acid substitutions may be made at one or more nonessential amino acid residues. A “nonessential” amino acid residue is a residue that can be altered from the reference sequence of a polypeptide without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be made in nonconserved regions that retain function. In general, such substitutions would not be made for conserved amino acid residues, or for amino acid residues residing within a conserved motif, where such residues are essential for polypeptide activity. However, one of skill in the art would understand that functional variants may have minor conserved or nonconserved alterations in the conserved residues.

Antibodies to the HPPR and/or HPPD enzymes of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; U.S. Pat. No. 4,196,265).

Thus, one aspect of the invention concerns antibodies, single-chain antigen binding molecules, or other proteins that specifically bind to one or more of the protein or peptide molecules of the invention and their homologs, fusions or fragments. In a particularly preferred embodiment, the antibody specifically binds to a protein having the amino acid sequence set forth in SEQ ID NO:3-59 and 78-88or a fragment thereof. In another embodiment, the antibody specifically binds to a fusion protein comprising an amino acid sequence selected from the amino acid sequence set forth in SEQ ID NO:3-59 and 78-88, or a fragment thereof. In some embodiments, the antibody specifically binds to the region of the protein corresponding to amino acid position 178 of SEQ ID NO:1, or the region of the protein corresponding to amino acid position 188 of SEQ ID NO:1, or the region of the protein corresponding to amino acid position 200 of SEQ ID NO:1, or the region of the protein corresponding to amino acid position 226 of SEQ ID NO:1, or the region of the protein corresponding to amino acid positions 335-340 of SEQ ID NO:1. In other embodiments, the antibody specifically binds to the region of the protein corresponding to amino acid position 193 of SEQ ID NO:59, or the region of the protein corresponding to amino acid position 209 of SEQ ID NO:59, or the region of the protein corresponding to amino acid position 221 of SEQ ID NO:59, or the region of the protein corresponding to amino acid position 247 of SEQ ID NO:59, or the region of the protein corresponding to amino acid positions 351-356 of SEQ ID NO:59.

Antibodies of the invention may be used to quantitatively or qualitatively detect the protein or peptide molecules of the invention, or to detect post translational modifications of the proteins. As used herein, an antibody or peptide is said to “specifically bind” to a protein or peptide molecule of the invention if such binding is not competitively inhibited by the presence of non-related molecules.

F. Gene Stacking

In the commercial production of crops, it is desirable to eliminate under reliable pesticidal management unwanted plants (i.e., “weeds”) from a field of crop plants. An ideal treatment would be one which could be applied to an entire field but which would eliminate only the unwanted plants while leaving the crop plants unaffected. One such treatment system would involve the use of crop plants which are tolerant to an herbicide so that when the herbicide is sprayed on a field of herbicide-tolerant crop plants, the crop plants would continue to thrive while non-herbicide-tolerant weeds are killed or severely damaged. Ideally, such treatment systems would take advantage of varying herbicide properties so that weed control could provide the best possible combination of flexibility and economy. For example, individual herbicides have different longevities in the field, and some herbicides persist and are effective for a relatively long time after they are applied to a field while other herbicides are quickly broken down into other and/or non-active compounds. An ideal treatment system would allow the use of different herbicides so that growers could tailor the choice of herbicides for a particular situation.

While a number of herbicide-tolerant crop plants are presently commercially available, an issue that has arisen for many commercial herbicides and herbicide/crop combinations is that individual herbicides typically have incomplete spectrum of activity against common weed species. For most individual herbicides which have been in use for some time, populations of herbicide resistant weed species and biotypes have become more prevalent (see, e.g., Tranel and Wright (2002) Weed Science 50: 700-712; Owen and Zelaya (2005) Pest Manag. Sci. 61: 301-311). Transgenic plants which are tolerant to more than one herbicide have been described (see, e.g., W02005/012515). However, improvements in every aspect of crop production, weed control options, extension of residual weed control, and improvement in crop yield are continuously in demand.

Silencing of the HPPR genes of the invention can be advantageously combined with expression of one or more HPPD protein or nucleotide sequences. In addition, HPPR silencing+HPPD expression is advantageously combined in plants with other genes which encode proteins or RNAs that confer useful agronomic properties to such plants. Among the genes which encode proteins or RNAs that confer useful agronomic properties on the transformed plants, mention can be made of the DNA sequences encoding proteins which confer tolerance to one or more herbicides that, according to their chemical structure, differ from HPPD inhibitor herbicides, and others which confer tolerance to certain insects, those which confer tolerance to certain diseases, DNAs that encodes RNAs that provide nematode or insect control, and the like.

Such genes are in particular described in published PCT Patent Applications WO91/02071 and WO95/06128 and in U.S. Pat. No. 7,923,602 and US Patent Application Publication No. 20100166723, each of which is herein incorporated by reference in its entirety.

Among the DNA sequences encoding proteins which confer tolerance to certain herbicides on the transformed plant cells and plants, mention can be made of a bar or PAT gene or the Streptomyces coelicolor gene described in WO2009/152359 which confers tolerance to glufosinate herbicides, a gene encoding a suitable EPSPS which confers tolerance to herbicides having EPSPS as a target, such as glyphosate and its salts (U.S. Pat. Nos. 4,535,060, 4,769,061, 5,094,945, 4,940,835, 5,188,642, 4,971,908, 5,145,783, 5,310,667, 5,312,910, 5,627,061, 5,633,435), a gene encoding glyphosate-n-acetyltransferase (for example, U.S. Pat. Nos. 8,222,489, 8,088,972, 8,044,261, 8,021,857, 8,008,547, 7,999,152, 7,998,703, 7,863,503, 7,714,188, 7,709,702, 7,666,644, 7,666,643, 7,531,339, 7,527,955, and 7,405,074), or a gene encoding glyphosate oxidoreductase (for example, U.S. Pat. No. 5,463,175).

Among the DNA sequences encoding a suitable EPSPS which confer tolerance to the herbicides which have EPSPS as a target, mention will more particularly be made of the gene which encodes a plant EPSPS, in particular maize EPSPS, particularly a maize EPSPS which comprises two mutations, particularly a mutation at amino acid position 102 and a mutation at amino acid position 106 (WO2004/074443), and which is described in Patent Application U.S. Pat. No. 6,566,587, hereinafter named double mutant maize EPSPS or 2mEPSPS, or the gene which encodes an EPSPS isolated from Agrobacterium and which is described by sequence ID No. 2 and sequence ID No. 3 of U.S. Pat. No. 5,633,435, also named CP4.

Among the DNA sequences encoding a suitable EPSPS which confer tolerance to the herbicides which have EPSPS as a target, mention will more particularly be made of the gene which encodes an EPSPS GRG23 from Arthrobacter globiformis, but also the mutants GRG23 ACE1, GRG23 ACE2, or GRG23 ACE3, particularly the mutants or variants of GRG23 as described in WO2008/100353, such as GRG23(ace3)R173K of SEQ ID No. 29 in WO2008/100353.

In the case of the DNA sequences encoding EPSPS, and more particularly encoding the above genes, the sequence encoding these enzymes is advantageously preceded by a sequence encoding a transit peptide, in particular the “optimized transit peptide” described in U.S. Pat. Nos. 5,510,471 or 5,633,448.

Exemplary herbicide tolerance traits that can be combined with the nucleic acid sequence of the invention further include at least one ALS (acetolactate synthase) inhibitor (WO2007/024782); a mutated Arabidopsis ALS/AHAS gene (U.S. Pat. No. 6,855,533); genes encoding 2,4-D-monooxygenases conferring tolerance to 2,4-D (2,4-dichlorophenoxyacetic acid) by metabolization (U.S. Pat. No. 6,153,401); and genes encoding Dicamba monooxygenases conferring tolerance to dicamba (3,6-dichloro-2-methoxybenzoic acid) by metabolization (US 2008/0119361 and US 2008/0120739).

In various embodiments, the HPPR and/or HPPD of the invention is stacked with one or more herbicide tolerant genes, including one or more additional HPPD inhibitor herbicide tolerant genes, and/or one or more genes tolerant to glyphosate and/or glufosinate. In one embodiment, the HPPR and/or HPPD of the invention is combined with 2mEPSPS and bar.

Among the DNA sequences encoding proteins concerning properties of tolerance to insects, mention will more particularly be made of the Bt proteins widely described in the literature and well known to those skilled in the art. Mention will also be made of proteins extracted from bacteria such as Photorhabdus (WO97/17432 & WO98/08932).

Among such DNA sequences encoding proteins of interest which confer novel properties of tolerance to insects, mention will more particularly be made of the Bt Cry or VIP proteins widely described in the literature and well known to those skilled in the art. These include the Cry1F protein or hybrids derived from a Cry1F protein (e.g., the hybrid Cry1A-Cry1F proteins described in U.S. Pat. Nos. 6,326,169; 6,281,016; 6,218,188, or toxic fragments thereof), the Cry1A-type proteins or toxic fragments thereof, preferably the Cry1Ac protein or hybrids derived from the Cry1Ac protein (e.g., the hybrid Cry1Ab-Cry1Ac protein described in U.S. Pat. No. 5,880,275) or the Cry1Ab or Bt2 protein or insecticidal fragments thereof as described in EP451878, the Cry2Ae, Cry2Af or Cry2Ag proteins as described in WO2002/057664 or toxic fragments thereof, the Cry1A.105 protein described in WO 2007/140256 (SEQ ID No. 7) or a toxic fragment thereof, the VIP3Aa19 protein of NCBI accession ABG20428, the VIP3Aa20 protein of NCBI accession ABG20429 (SEQ ID No. 2 in WO 2007/142840), the VIP3A proteins produced in the COT202 or COT203 cotton events (WO2005/054479 and WO2005/054480, respectively), the Cry proteins as described in WO2001/47952, the VIP3Aa protein or a toxic fragment thereof as described in Estruch et al. (1996), Proc Natl Acad Sci USA. 28; 93(11):5389-94 and U.S. Pat. No. 6,291,156, the insecticidal proteins from Xenorhabdus (as described in WO98/50427), Serratia (particularly from S. entomophila) or Photorhabdus species strains, such as Tc-proteins from Photorhabdus as described in WO98/08932 (e.g., Waterfield et al., 2001, Appl Environ Microbiol. 67(11):5017-24; Ffrench-Constant and Bowen, 2000, Cell Mol Life Sci.; 57(5):828-33). Also any variants or mutants of any one of these proteins differing in some (1-10, preferably 1-5) amino acids from any of the above sequences, particularly the sequence of their toxic fragment, or which are fused to a transit peptide, such as a plastid transit peptide, or another protein or peptide, is included herein.

In various embodiments, the HPPR and/or HPPD sequences of the invention can be combined in plants with one or more genes conferring a desirable trait, such as herbicide tolerance, insect tolerance, drought tolerance, nematode control, water use efficiency, nitrogen use efficiency, improved nutritional value, disease resistance, improved photosynthesis, improved fiber quality, stress tolerance, improved reproduction, and the like.

Particularly useful transgenic events which may be combined with the genes of the current invention in plants of the same species (e.g., by crossing or by re-transforming a plant containing another transgenic event with a chimeric gene of the invention), include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control—herbicide tolerance, deposited as PTA-6233, described in WO2005/103266 or US-A 2005-216969); Event 3006-210-23 (cotton, insect control—herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control—herbicide tolerance, deposited as ATCC PTA-11508, described in WO 11/075593); Event 43A47 (corn, insect control—herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480);); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control—herbicide tolerance, deposited as ATCC PTA 11384, described in US-A 2006-070139); Event DAS-59132 (corn, insect control—herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-1 (brinjal, insect control, not deposited, described in WO 07/091277); Event FI117 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO 98/044140); Event GHB119 (cotton, insect control—herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN1 (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control—herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MS11 (oilseed rape, pollination control—herbicide tolerance, deposited as ATCC PTA-850 or PTA-2485, described in WO2001/031042); Event MS8 (oilseed rape, pollination control—herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control—herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control—herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control—herbicide tolerance, not deposited, described in US-A 2005-039226 or WO2004/099447); Event VIP1034 (corn, insect control—herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession N° PTA-11041) optionally stacked with event EE-GM1/LL27 or event EE-GM2/LL55 (WO2011/063413A2), event DAS-68416-4 (soybean, herbicide tolerance, ATCC Accession N° PTA-10442, WO2011/066360A1), event DAS-68416-4 (soybean, herbicide tolerance, ATCC Accession N° PTA-10442, WO2011/066384A1), event DP-040416-8 (corn, insect control, ATCC Accession N° PTA-11508, WO2011/075593A1), event DP-043A47-3 (corn, insect control, ATCC Accession N° PTA-11509, WO2011/075595A1), event DP-004114-3 (corn, insect control, ATCC Accession N° PTA-11506, WO2011/084621A1), event DP-032316-8 (corn, insect control, ATCC Accession N° PTA-11507, WO2011/084632A1), event MON-88302-9 (oilseed rape, herbicide tolerance, ATCC Accession N° PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession N°. PTA-10296, WO2012/051199A2), event DAS-44406-6 (soybean, stacked herbicide tolerance, ATCC Accession N°. PTA-11336, WO2012/075426A1), event DAS-14536-7 (soybean, stacked herbicide tolerance, ATCC Accession N°. PTA-11335, WO2012/075429A1), event SYN-000H2-5 (soybean, herbicide tolerance, ATCC Accession N°. PTA-11226, WO2012/082548A2), event DP-061061-7 (oilseed rape, herbicide tolerance, no deposit N° available, WO2012071039A1), event DP-073496-4 (oilseed rape, herbicide tolerance, no deposit N° available, US2012131692), event 8264.44.06.1 (soybean, stacked herbicide tolerance, Accession N° PTA-11336, WO2012075426A2), event 8291.45.36.2 (soybean, stacked herbicide tolerance, Accession N°. PTA-11335, WO2012075429A2), event SYHT0H2 (soybean, ATCC Accession N°. PTA-11226, WO2012/082548A2), event MON88701 (cotton, ATCC Accession N° PTA-11754, WO2012/134808A1), event KK179-2 (alfalfa, ATCC Accession N° PTA-11833, WO2013/003558A1), event pDAB8264.42.32.1 (soybean, stacked herbicide tolerance, ATCC Accession N° PTA-11993, WO2013/010094A1), event MZDTO9Y (corn, ATCC Accession N° PTA-13025, WO2013/012775A1).

G. Polynucleotide Constructs

The polynucleotides constructed to silence the HPPR genes of the present invention and/or encoding the HPPD polypeptides of the present invention may be modified to obtain or enhance expression in plant cells. The polynucleotides encoding the polypeptides identified herein may be provided in expression cassettes for expression in the plant of interest. A “plant expression cassette” includes a DNA construct, including a recombinant DNA construct, that is capable of resulting in the expression of a polynucleotide in a plant cell. The cassette can include in the 5′-3′ direction of transcription, a transcriptional initiation region (i.e., promoter, particularly a heterologous promoter) operably-linked to one or more polynucleotides of interest, and/or a translation and transcriptional termination region (i.e., termination region) functional in plants. The cassette may additionally contain at least one additional polynucleotide to be introduced into the organism, such as a selectable marker gene. Alternatively, the additional polynucleotide(s) can be provided on multiple expression cassettes. Such an expression cassette is provided with a plurality of restriction sites for insertion of the polynucleotide(s) to be under the transcriptional regulation of the regulatory regions.

In a further embodiment, the present invention relates to a chimeric gene comprising a coding sequence comprising heterologous the nucleic acid of the invention operably linked to a plant-expressible promoter and optionally a transcription termination and polyadenylation region. “Heterologous” generally refers to the polynucleotide or polypeptide that is not endogenous to the cell or is not endogenous to the location in the native genome in which it is present, and has been added to the cell by infection, transfection, microinjection, electroporation, microprojection, or the like. By “operably linked” is intended a functional linkage between two polynucleotides. For example, when a promoter is operably linked to a DNA sequence, the promoter sequence initiates and mediates transcription of the DNA sequence. It is recognized that operably linked polynucleotides may or may not be contiguous and, where used to reference the joining of two polypeptide coding regions, the polypeptides are expressed in the same reading frame.

The promoter may be any polynucleotide sequence which shows transcriptional activity in the chosen plant cells, plant parts, or plants. The promoter may be native or analogous, or foreign or heterologous, to the plant host and/or to the DNA sequence of the invention. Where the promoter is “native” or “analogous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds (1987) Nucleic Acids Res. 15:2343-2361. Also, the location of the promoter relative to the transcription start may be optimized. See, e.g., Roberts et al. (1979) Proc. Natl. Acad. Sci. USA, 76:760-764. Many suitable promoters for use in plants are well known in the art.

For instance, suitable constitutive promoters for use in plants include: the promoters from plant viruses, such as the peanut chlorotic streak caulimovirus (PC1SV) promoter (U.S. Pat. No. 5,850,019); the 35S promoter from cauliflower mosaic virus (CaMV) (Odell et al. (1985) Nature 313:810-812); promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171 and U.S. Pat. No. 5,641,876); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730 and U.S. Pat. No. 5,510,474); maize H3 histone (Lepetit et al. (1992) Mol. Gen. Genet. 231:276-285 and Atanassova et al. (1992) Plant J. 2(3):291-300); Brassica napus ALS3 (PCT application WO97/41228); a plant ribulose-biscarboxylase/oxygenase (RuBisCO) small subunit gene; the circovirus (AU 689 311) or the Cassava vein mosaic virus (CsVMV, U.S. Pat. No. 7,053,205); and promoters of various Agrobacterium genes (see U.S. Pat. Nos. 4,771,002; 5,102,796; 5,182,200; and 5,428,147).

Suitable inducible promoters for use in plants include: the promoter from the ACE1 system which responds to copper (Mett et al. (1993) PNAS 90:4567-4571); the promoter of the maize In2 gene which responds to benzenesulfonamide herbicide safeners (Hershey et al. (1991) Mol. Gen. Genetics 227:229-237 and Gatz et al. (1994) Mol. Gen. Genetics 243:32-38); and the promoter of the Tet repressor from Tn10 (Gatz et al. (1991) Mol. Gen. Genet. 227:229-237). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al. (1991) Proc. Natl. Acad. Sci. USA 88:10421) or the recent application of a chimeric transcription activator, XVE, for use in an estrogen receptor-based inducible plant expression system activated by estradiol (Zuo et al. (2000) Plant J., 24:265-273). Other inducible promoters for use in plants are described in EP 332104, PCT WO 93/21334 and PCT WO 97/06269 which are herein incorporated by reference in their entirety. Promoters composed of portions of other promoters and partially or totally synthetic promoters can also be used. See, e.g., Ni et al. (1995) Plant J. 7:661-676 and PCT WO 95/14098 describing such promoters for use in plants.

In one embodiment of this invention, a promoter sequence specific for particular regions or tissues of plants can be used to silence the HPPR genes and/or express the HPPD proteins of the invention, such as promoters specific for seeds (Datla, R. et al., 1997, Biotechnology Ann. Rev. 3, 269-296), especially the napin promoter (EP 255 378 A1), the phaseolin promoter, the glutenin promoter, the helianthinin promoter (WO92/17580), the albumin promoter (WO98/45460), the oleosin promoter (WO98/45461), the SAT1 promoter or the SAT3 promoter (PCT/US98/06978).

Use may also be made of an inducible promoter advantageously chosen from the phenylalanine ammonia lyase (PAL), HMG-CoA reductase (HMG), chitinase, glucanase, proteinase inhibitor (PI), PR1 family gene, nopaline synthase (nos) and vspB promoters (U.S. Pat. No. 5,670,349, Table 3), the HMG2 promoter (U.S. Pat. No. 5,670,349), the apple beta-galactosidase (ABG1) promoter and the apple aminocyclopropane carboxylate synthase (ACC synthase) promoter (WO98/45445). Multiple promoters can be used in the constructs of the invention, including in succession.

The promoter may include, or be modified to include, one or more enhancer elements. In some embodiments, the promoter may include a plurality of enhancer elements. Promoters containing enhancer elements provide for higher levels of transcription as compared to promoters that do not include them. Suitable enhancer elements for use in plants include the PC1SV enhancer element (U.S. Pat. No. 5,850,019), the CaMV 35S enhancer element (U.S. Pat. Nos. 5,106,739 and 5,164,316) and the FMV enhancer element (Maiti et al. (1997) Transgenic Res. 6:143-156); the translation activator of the tobacco mosaic virus (TMV) described in Application WO87/07644, or of the tobacco etch virus (TEV) described by Carrington & Freed 1990, J. Virol. 64: 1590-1597, for example, or introns such as the adh1 intron of maize or intron 1 of rice actin. See also PCT WO96/23898, WO2012/021794, WO2012/021797, WO2011/084370, and WO2011/028914.

Often, such constructs can contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. By “signal sequence” is intended a sequence that is known or suspected to result in co-translational or post-translational peptide transport across the cell membrane. In eukaryotes, this typically involves secretion into the Golgi apparatus, with some resulting glycosylation. By “leader sequence” is intended any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. It may also be preferable to engineer the plant expression cassette to contain an intron, such that mRNA processing of the intron is required for expression.

By “3′ untranslated region” is intended a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. By “5′ untranslated region” is intended a polynucleotide located upstream of a coding sequence.

Other upstream or downstream untranslated elements include enhancers. Enhancers are polynucleotides that act to increase the expression of a promoter region. Enhancers are well known in the art and include, but are not limited to, the SV40 enhancer region and the 35S enhancer element.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; Joshi et al. (1987) Nucleic Acid Res. 15:9627-9639; and European Patent Application EP 0 633 317 A1.

In one aspect of the invention, synthetic DNA sequences are designed for a given polypeptide, such as the polypeptides of the invention. Expression of the open reading frame of the synthetic DNA sequence in a cell results in production of the polypeptide of the invention. Synthetic DNA sequences can be useful to simply remove unwanted restriction endonuclease sites, to facilitate DNA cloning strategies, to alter or remove any potential codon bias, to alter or improve GC content, to remove or alter alternate reading frames, and/or to alter or remove intron/exon splice recognition sites, polyadenylation sites, Shine-Delgarno sequences, unwanted promoter elements and the like that may be present in a native DNA sequence. It is also possible that synthetic DNA sequences may be utilized to introduce other improvements to a DNA sequence, such as introduction of an intron sequence, creation of a DNA sequence that in expressed as a protein fusion to organelle targeting sequences, such as chloroplast transit peptides, apoplast/vacuolar targeting peptides, or peptide sequences that result in retention of the resulting peptide in the endoplasmic reticulum. Synthetic genes can also be synthesized using host cell-preferred codons for improved expression, or may be synthesized using codons at a host-preferred codon usage frequency. See, for example, Campbell and Gowri (1990) Plant Physiol. 92:1-11; U.S. Pat. Nos. 6,320,100; 6,075,185; 5,380,831; and 5,436,391, U.S. Published Application Nos. 20040005600 and 20010003849, and Murray et al. (1989) Nucleic Acids Res. 17:477-498, herein incorporated by reference.

In one embodiment, the polynucleotides of interest are targeted to the chloroplast for expression. In this manner, where the polynucleotide of interest is not directly inserted into the chloroplast, the expression cassette will additionally contain a polynucleotide encoding a transit peptide to direct the nucleotide of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See, for example, U.S. Pat. No. 5,380,831, herein incorporated by reference.

This plant expression cassette can be inserted into a plant transformation vector. By “transformation vector” is intended a DNA molecule that allows for the transformation of a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a polynucleotide construct designed for transfer between different host cells. “Expression vector” refers to a vector that has the ability to incorporate, integrate and express heterologous DNA sequences or fragments in a foreign cell.

The plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “polynucleotide of interest” (a polynucleotide engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science, 5:446-451). Several types of Agrobacterium strains (e.g., LBA4404, GV3101, EHA101, EHA105, etc.) can be used for plant transformation. The second plasmid vector is not necessary for introduction of polynucleotides into plants by other methods such as microprojection, microinjection, electroporation, polyethylene glycol, etc.

H. Plant Transformation

Methods of the invention involve introducing a nucleotide construct into a plant. By “introducing” is intended to present to the plant the nucleotide construct in such a manner that the construct gains access to the interior of a cell of the plant. The methods of the invention do not require that a particular method for introducing a nucleotide construct to a plant is used, only that the nucleotide construct gains access to the interior of at least one cell of the plant. Methods for introducing nucleotide constructs into plants are known in the art including, but not limited to, stable transformation methods, transient transformation methods, and virus-mediated methods. See, for example, the methods for transforming plant cells and regenerating plants described in: U.S. Pat. Nos. 4,459,355, 4,536,475, 5,464,763, 5,177,010, 5,187,073, EP 267,159 A1, EP 604 662 A1, EP 672 752 A1, U.S. Pat. Nos. 4,945,050, 5,036,006, 5,100,792, 5,371,014, 5,478,744, 5,179,022, 5,565,346, 5,484,956, 5,508,468, 5,538,877, 5,554,798, 5,489,520, 5,510,318, 5,204,253, 5,405,765, EP 442 174 A1, EP 486 233 A1, EP 486 234 A1, EP 539 563 A1, EP 674 725 A1, WO91/02071, WO95/06128, and WO2011/095460, each of which is herein incorporated by reference, particularly with respect to the transformation methods described therein.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into mature plants and produce fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Molecular and biochemical methods can be used to confirm the presence of the integrated heterologous gene of interest in the genome of transgenic plant.

Generation of transgenic plants may be performed by one of several methods, including, but not limited to, introduction of heterologous DNA by Agrobacterium into plant cells (Agrobacterium-mediated transformation), bombardment of plant cells with heterologous foreign DNA adhered to particles, and various other non-particle direct-mediated methods (e.g. Hiei et al. (1994) The Plant Journal 6:271-282; Ishida et al. (1996) Nature Biotechnology 14:745-750; Ayres and Park (1994) Critical Reviews in Plant Science 13:219-239; Bommineni and Jauhar (1997) Maydica 42:107-120) to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The plant cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome. In various embodiments, the seed can be coated with at least one fungicide and/or at least one insecticide, at least one herbicide, and/or at least one safener, or any combination thereof.

I. Evaluation of Plant Transformation

Following introduction of heterologous foreign DNA into plant cells, the transformation or integration of the heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual (Cold Spring Harbor Laboratory Press, Cold Spring Harbor, N.Y.)). PCR is carried out using oligonucleotide primers specific to the gene of interest or Agrobacterium vector background, etc.

Plant transformation may be confirmed by Southern blot analysis of genomic DNA (Sambrook and Russell (2001) supra). In general, total DNA is extracted from the transformant, digested with appropriate restriction enzymes, fractionated in an agarose gel and transferred to a nitrocellulose or nylon membrane. The membrane or “blot” can then be probed with, for example, radiolabeled ³²P target DNA fragment to confirm the integration of the introduced gene in the plant genome according to standard techniques (Sambrook and Russell, 2001, supra).

In Northern analysis, RNA is isolated from specific tissues of transformant, fractionated in a formaldehyde agarose gel, and blotted onto a nylon filter according to standard procedures that are routinely used in the art (Sambrook and Russell (2001) supra). Expression of RNA encoded by nucleotide sequences of the invention is then tested by hybridizing the filter to a radioactive probe derived from a GDC by methods known in the art (Sambrook and Russell (2001) supra). RNA can also be detected and/or quantified using reverse transcriptase PCR as known in the art (e.g., Green and Sambrook (2012) Molecular Cloning: A Laboratory Manual, 4^{th} Edition, Cold Spring Harbor Laboratory Press, Woodbury, N.Y.).

Western blot, ELISA, lateral flow testing, and biochemical assays and the like may be carried out on the transgenic plants to determine the presence of protein encoded by the herbicide tolerance gene by standard procedures (Sambrook and Russell (2001) supra) using antibodies that bind to one or more epitopes present on the herbicide tolerance protein.

In one aspect of the invention, the HPPR and/or HPPD genes described herein are useful as markers to assess transformation of bacterial or plant cells.

J. Use as a Marker for Transformation

The invention also relates to the use, in a method for transforming plants, of a nucleic acid which encodes an HPPD according to the invention as a marker gene or as a coding sequence which makes it possible to confer to the plant tolerance to herbicides which are HPPD inhibitors, and the use of one or more HPPD inhibitor(s) on plants comprising a nucleic acid sequence encoding a HPPD according to the invention. See, for example, U.S. Pat. No. 6,791,014, which is herein incorporated by reference in its entirety.

In this embodiment, an HPPD inhibitor can be introduced into the culture medium of the competent plant cells so as to bleach said cells before the transformation step. The bleached competent cells are then transformed with the gene for tolerance to HPPD inhibitors, as a selection marker, and the transformed cells which have integrated said selection marker into their genome become green, enabling them to be selected. Such a process makes it possible to decrease the time required for selecting the transformed cells.

Thus, one embodiment of the present invention consists of a method for transforming plant cells by introducing a heterologous gene into said plant cells with a gene for tolerance to HPPD inhibitors as selection markers, wherein the method comprises preparing and culturing competent plant cells capable of receiving the heterologous gene in a suitable medium and introducing a suitable amount of HPPD inhibitor into the suitable culture medium of the competent plant cells. The competent cells are then transformed with the heterologous gene and the selection marker, and the transformed cells comprising the heterologous gene are grown in a suitable medium and transformants selected therefrom. The transformed cells can then be regenerated into a fertile transformed plant.

K. Plants and Plant Parts

By “plant” is intended whole plants, plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). The present invention may be used for introduction of polynucleotides into any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Crop plants are also of interest, including, for example, maize, sorghum, wheat, sunflower, tomato, crucifers, peppers, potato, cotton, rice, soybean, sugarbeet, sugarcane, tobacco, barley, oilseed rape, etc.

This invention is suitable for any member of the monocot plant family including, but not limited to, maize, rice, barley, oats, wheat, sorghum, rye, sugarcane, pineapple, yams, onion, banana, coconut, and dates.

L. Methods for Increasing Plant Yield

Methods for increasing plant yield are provided. The methods comprise providing a plant comprising, or introducing into a plant or plant cell, a polynucleotide comprising a nucleotide sequence silencing an HPPR gene of the invention and/or encoding an HPPD of the invention, growing the plant or a seed thereof in a field, and producing a harvest from said plants or seeds. As defined herein, the “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase.

In specific methods, the plant comprising an HPPR-silencing sequence and/or HPPD sequence of the invention is treated with an effective concentration of an HPPD inhibitor herbicide, such as one or more HPPD inhibitor herbicide(s) selected from the group consisting of HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), and 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone, where the herbicide application results in enhanced plant yield.

Methods for conferring herbicide tolerance in a plant or plant part are also provided. In such methods, a nucleotide sequence silencing an HPPR gene of the invention and/or encoding an HPPD of the invention is introduced into the plant, wherein expression of the polynucleotide results in HPPD inhibitor herbicide tolerance. Plants produced via this method can be treated with an effective concentration of an herbicide (such as one or more HPPD inhibitor herbicide(s) selected from the group consisting of HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone) and display an increased tolerance to the herbicide. An “effective concentration” of an herbicide in this application is an amount sufficient to slow or stop the growth of plants or plant parts that are not naturally tolerant or rendered tolerant to the herbicide.

M. Methods of Controlling Weeds in a Field

The present invention therefore also relates to a method of controlling undesired plants or for regulating the growth of plants in crops of plants comprising a nucleotide sequence silencing an HPPR gene according to the invention and/or encoding an HPPD according to the invention, where one or more HPPD inhibitor herbicides, for example, one or more HPPD inhibitor herbicides selected from the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), and 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone, are applied to the plants (for example harmful plants such as monocotyledonous or dicotyledonous weeds or undesired crop plants), to the seeds (for example grains, seeds or vegetative propagules such as tubers or shoot parts with buds) or to the area on which the plants grow (for example the area under cultivation). In this context, an effective concentration of one or more HPPD inhibitor herbicide(s), for example, one or more HPPD inhibitor herbicides selected from the group consisting of HPPD inhibitor herbicides of the class of N (1,2,5-oxadiazol-3-yl)benzamides; N-(tetrazol-4-yl)- or N-(triazol-3-yl)arylcarboxamides, preferably such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), and 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone, the class of isoxazoles preferably such as isoxaflutole, or of the class of pyrazolinates, preferably such as pyrasulfotole and topramezone, particularly selected from tembotrione, sulcotrione, topramezone, bicyclopyrone, tefuryltrione, isoxaflutole, and mesotrione, can be applied for example pre-planting (if appropriate also by incorporation into the soil), pre-emergence or post-emergence, and may be combined with the application of other herbicides to which the crop is naturally tolerant, or to which it is resistant via expression of one or more other herbicide resistance transgenes. See, e.g., U.S. App. Pub. No. 2004/0058427 and PCT App. Pub. No. WO98/20144. By “effective concentration” is intended the concentration which controls the growth or spread of weeds or other untransformed plants without significantly affecting the HPPD inhibitor-tolerant plant or plant seed. Those of skill in the art understand that application of herbicides can take many different forms and can take place at many different times prior to and/or throughout the seed planting and growth process. “Pre-emergent” application refers to an herbicide which is applied to an area of interest (e.g., a field or area of cultivation) before a plant emerges visibly from the soil. “Post-emergent” application refers to an herbicide which is applied to an area after a plant emerges visibly from the soil. In some instances, the terms “pre-emergent” and “post-emergent” are used with reference to a weed in an area of interest, and in some instances these terms are used with reference to a crop plant in an area of interest. When used with reference to a weed, these terms may apply to a particular type of weed or species of weed that is present or believed to be present in the area of interest. “Pre-plant incorporation” of an herbicide involves the incorporation of compounds into the soil prior to planting.

Thus, the present invention comprises a method of controlling weeds in a field comprising planting in a field a plant or a seed thereof in which one or more HPPR genes are silenced, optionally comprising an HPPD of the invention, and applying to said plant or area surrounding said plant an effective concentration of one or more HPPD inhibitor herbicides.

In one embodiment of this invention, a field to be planted with plants (such as soybean, cotton, corn, or wheat plants, e.g.) containing an HPPR and/or HPPD nucleotide sequence of the invention, can be treated with an HPPD inhibitor herbicide, such as isoxaflutole (IFT), before the plants are planted or the seeds are sown, which cleans the field of weeds that are killed by the HPPD inhibitor, allowing for no-till practices, followed by planting or sowing of the plants in that same pre-treated field later on (burndown application using an HPPD inhibitor herbicide). The residual activity of IFT will also protect the emerging and growing plants from competition by weeds in the early growth stages. Once the plants have a certain size, and weeds tend to re-appear, glufosinate or glyphosate, or an HPPD inhibitor or a mixture of an HPPD inhibitor with another herbicide such as glyphosate, can be applied as post-emergent herbicide over the top of the plants, when such plants are tolerant to said herbicides.

In another embodiment of this invention, a field in which seeds containing an HPPR and/or HPPD nucleotide sequence of the invention were sown, can be treated with an HPPD inhibitor herbicide, such as IFT, before the plants emerge but after the seeds are sown (the field can be made weed-free before sowing using other means, typically conventional tillage practices such as ploughing, chissel ploughing, or seed bed preparation), where residual activity will keep the field free of weeds killed by the herbicide so that the emerging and growing plants have no competition by weeds (pre-emergence application of an HPPD inhibitor herbicide). Once the plants have a certain size, and weeds tend to re-appear, glufosinate or glyphosate, or an HPPD inhibitor or a mixture of an HPPD inhibitor with another herbicide such as glyphosate, can be applied as post-emergent herbicide over the top of the plants, when such plants are tolerant to said herbicides.

In another embodiment of this invention, plants containing an HPPR and/or HPPD nucleotide sequence of the invention, can be treated with an HPPD inhibitor herbicide, over the top of the plants that have emerged from the seeds that were sown, which cleans the field of weeds killed by the HPPD inhibitor, which application can be together with (e.g., in a spray tank mix), followed by or preceded by a treatment with glyphosate or glufosinate as post-emergent herbicide over the top of the plants (post-emergence application of an HPPD inhibitor herbicide (with or without glyphosate)), when such plants are tolerant to such herbicides.

Examples of individual representatives of the monocotyledonous and dicotyledonous weeds which can be controlled with an HPPD inhibitor herbicide include:
Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.
Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

HPPD inhibitor herbicides useful in the present invention, including but not limited to HPPD inhibitor herbicides of the class of N (1,2,5-oxadiazol-3-yl)benzamides; N-(tetrazol-4-yl)- or N-(triazol-3-yl)arylcarboxamides, such as 2-chloro-3-ethoxy-4-(methylsulfonyl)-N-(1-methyl-1H-tetrazol-5-yl)benzamide and 2-Chloro-3-(methoxymethyl)-4-(methylsulfonyl)-N-(1-methyl-1H-tetrazol-5-yl)benzamide; N-(1,3,4-oxadiazol-2-yl)benzamides, preferably such as 2-methyl-N-(5-methyl-1,3,4-oxadiazol-2-yl)-3-(methylsulfonyl)-4-(trifluoromethyl)benzamide (Cmpd. 1); N-(tetrazol-5-yl)- or N-(triazol-3-yl)arylcarboxamides, preferably such as 2-chloro-3-ethoxy-4-(methylsulfonyl)-N-(1-methyl-1H-tetrazol-5-yl)benzamide (Cmpd. 2), 4-(difluoromethyl)-2-methoxy-3-(methylsulfonyl)-N-(1-methyl-1H-tetrazol-5-yl)benzamide (Cmpd. 3), 2-chloro-3-(methylsulfanyl)-N-(1-methyl-1H-tetrazol-5-yl)-4-(trifluoromethyl)benzamide (Cmpd. 4), 2-(methoxymethyl)-3-(methylsulfinyl)-N-(1-methyl-1H-tetrazol-5-yl)-4-(trifluoromethyl)benzamide (Cmpd. 5); pyridazinone derivatives (WO2013/050421 and WO2013/083774); substituted 1,2,5-oxadiazoles (WO2013/072300 and WO2013/072402); and oxoprazin derivatives (WO2013/054495); triketones, preferably such as tembotrione, sulcotrione and mesotrione; the class of isoxazoles preferably such as isoxaflutole; or of the class of pyrazolinates, preferably such as pyrasulfotole and topramezone, can be formulated in various ways, depending on the prevailing biological and/or physico-chemical parameters. Examples of possible formulations are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for application by broadcasting and on the soil, granules (GR) in the form of microgranules, spray granules, coated granules and adsorption granules, water-dispersible granules (WG), water-soluble granules (SG), ULV formulations, microcapsules and waxes.

These individual types of formulation are known in principle and are described, for example, in: Winnacker-Kuchler, “Chemische Technologie” [Chemical technology], volume 7, C. Hanser Verlag Munich, 4th Ed. 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives, are also known and are described, for example, in: Watkins, “Handbook of Insecticide Dust Diluents and Carriers”, 2nd Ed., Darland Books, Caldwell N.J., H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflächenaktive Äthylenoxidaddukte” [Interface-active ethylene oxide adducts], Wiss. Verlagsgesell., Stuttgart 1976; Winnacker-Küchler, “Chemische Technologie” [Chemical technology], volume 7, C. Hanser Verlag Munich, 4th Ed. 1986.

Based on these formulations, it is also possible to prepare combinations with other pesticidally active substances such as, for example, insecticides, acaricides, herbicides, fungicides, and with safeners, fertilizers and/or growth regulators, for example in the form of a ready mix or a tank mix.

N. Methods of Introducing Gene of the Invention into Another Plant

Also provided herein are methods of introducing the HPPR and/or HPPD nucleotide sequence of the invention into another plant. The HPPR and/or HPPD nucleotide sequence of the invention, or a fragment thereof, can be introduced into second plant by recurrent selection, backcrossing, pedigree breeding, line selection, mass selection, mutation breeding and/or genetic marker enhanced selection.

Thus, in one embodiment, the methods of the invention comprise crossing a first plant comprising an HPPR-silencing and/or HPPD nucleotide sequence of the invention with a second plant to produce F1 progeny plants and selecting F1 progeny plants that are tolerant to an HPPD inhibitor herbicide or that comprise the HPPR-silencing and/or HPPD nucleotide sequence of the invention. The methods may further comprise crossing the selected progeny plants with the first plant comprising the HPPR-silencing and/or HPPD nucleotide sequence of the invention to produce backcross progeny plants and selecting backcross progeny plants that are tolerant to an HPPD inhibitor herbicide or that comprise the HPPR-silencing and/or HPPD nucleotide sequence of the invention. Methods for evaluating HPPD inhibitor herbicide tolerance are provided elsewhere herein. The methods may further comprise repeating these steps one or more times in succession to produce selected second or higher backcross progeny plants that are tolerant to an HPPD inhibitor herbicide or that comprise the HPPR-silencing and/or HPPD nucleotide sequence of the invention.

Any breeding method involving selection of plants for the desired phenotype can be used in the method of the present invention. In some embodiments, The F1 plants may be self-pollinated to produce a segregating F2 generation. Individual plants may then be selected which represent the desired phenotype (e.g., HPPD inhibitor herbicide tolerance) in each generation (F3, F4, F5, etc.) until the traits are homozygous or fixed within a breeding population.

The second plant can be a plant having a desired trait, such as herbicide tolerance, insect tolerance, drought tolerance, nematode control, water use efficiency, nitrogen use efficiency, improved nutritional value, disease resistance, improved photosynthesis, improved fiber quality, stress tolerance, improved reproduction, and the like. The second plant may be an elite event as described elsewhere herein

In various embodiments, plant parts (whole plants, plant organs (e.g., leaves, stems, roots, etc.), seeds, plant cells, propagules, embryos, and the like) can be harvested from the resulting cross and either propagated or collected for downstream use (such as food, feed, biofuel, oil, flour, meal, etc.).

O. Methods of Obtaining a Plant Product

The present invention also relates to a process for obtaining a commodity product, comprising harvesting and/or milling the grains from a crop comprising an HPPR and/or HPPD sequence of the invention to obtain the commodity product. Agronomically and commercially important products and/or compositions of matter including but not limited to animal feed, commodities, and plant products and by-products that are intended for use as food for human consumption or for use in compositions and commodities that are intended for human consumption, particularly devitalized seed/grain products, including a (semi-)processed products produced from such grain/seeds, wherein said product is or comprises whole or processed seeds or grain, animal feed, corn or soy meal, corn or soy flour, corn, corn starch, soybean meal, soy flour, flakes, soy protein concentrate, soy protein isolates, texturized soy protein concentrate, cosmetics, hair care products, soy nut butter, natto, tempeh, hydrolyzed soy protein, whipped topping, shortening, lecithin, edible whole soybeans (raw, roasted, or as edamame), soy yogurt, soy cheese, tofu, yuba, as well as cooked, polished, steamed, baked or parboiled grain, and the like are intended to be within the scope of the present invention if these products and compositions of matter contain detectable amounts of the nucleotide and/or amino acid sequences set forth herein as being diagnostic for any plant containing such nucleotide sequences.

The following examples are offered by way of illustration and not by way of limitation.

### Experimental

### EXAMPLE 1

To determine whether blocking 4-hydroxyphenylpyruvate reductase (HPPR) could increase tolerance to 4-hydroxyphenylpyruvate dioxygenase (HPPD) inhibitors by funneling more metabolites toward the production of HPPD, a RNAi construct designed to inhibit expression of two putative HPPR genes and co-express a mutant bacterial Pf-HPPD enzyme (Pf-HPPD-evo41; SEQ ID NO: 16) were used to transform soybean plant cells.

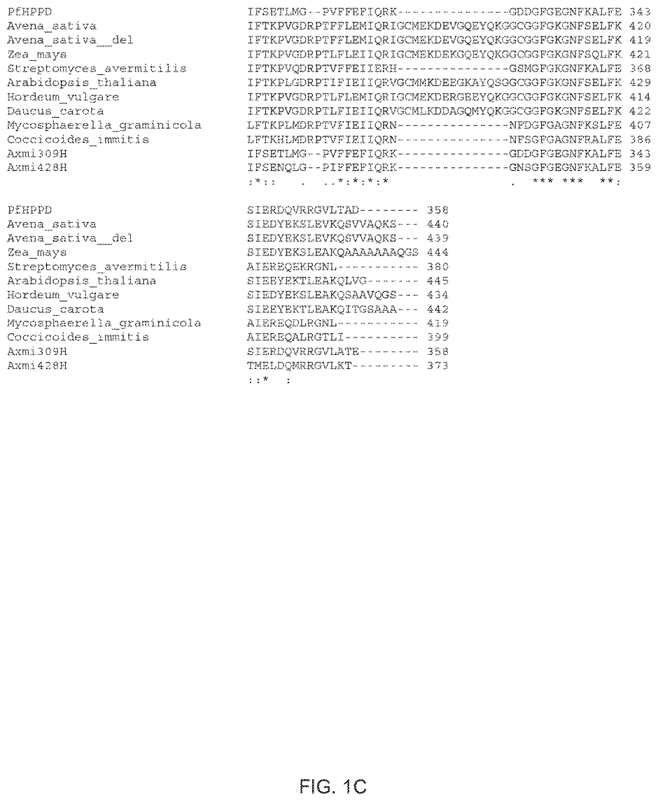

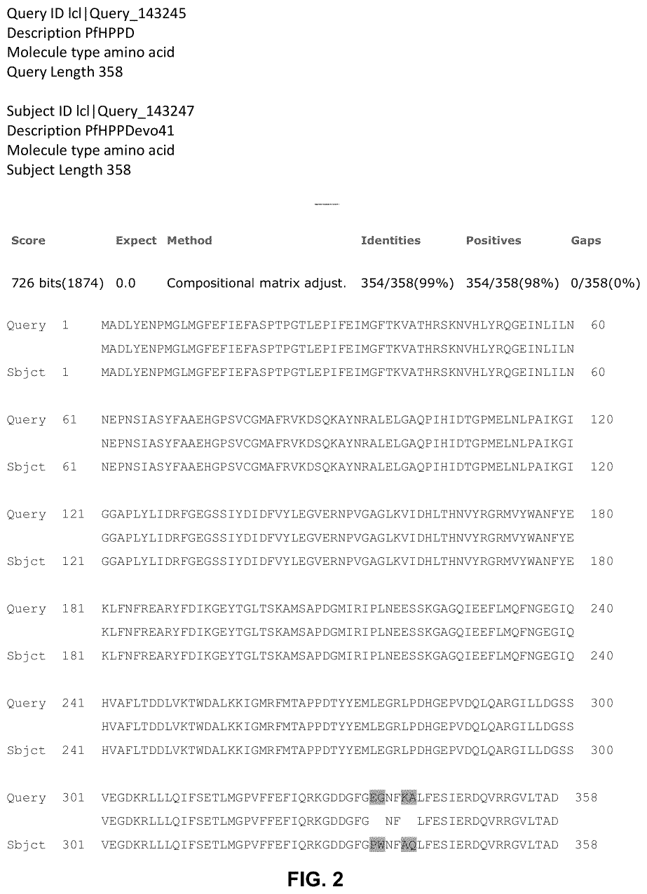

FIG. 2 shows an alignment of the amino acid between Pf-HPPD (Query) and Pf-HPPD-evo41 (Sbjct). Pf-HPPD-evo41 (SEQ ID NO:16) has a proline at the amino acid position corresponding to amino acid position 335, a tryptophan at the amino acid position corresponding to amino acid position 336, an alanine at the amino acid position corresponding to amino acid position 339, and a glutamine at the amino acid position corresponding to amino acid position 340 of Pf-HPPD (SEQ ID NO:1).

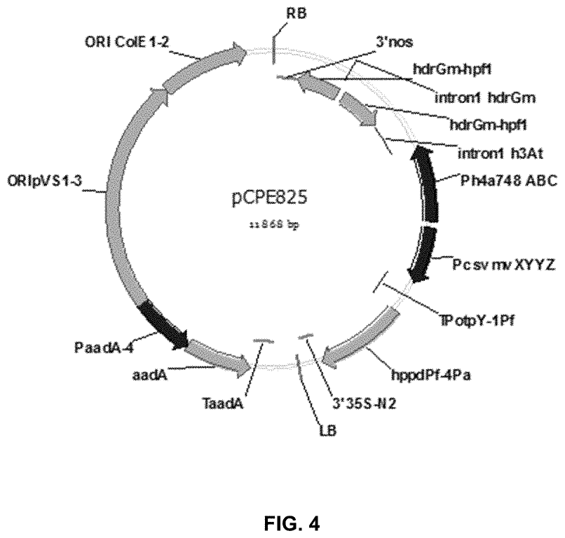

FIG. 4 shows a DNA construct (pCPE825) designed to silence the expression of the two putative soybean HPPR genes (SEQ ID NOs: 89 and 90) as well as to express Pf-HPPD-evo41 in soybean. pCPE825 includes a DNA coding sequence for Pf-HPPD-evo41 driven by a Cassava vein mosaic virus promoter (CsVMV) and a RNAi (hairpin) cassette designed to silence the endogenous putative soybean HPPRs (hdr-Gm). The RNAi cassette includes as operably linked components: (i) a sense polynucleotide strand comprising a concatemer of at least 20 contiguous nucleotides from SEQ ID NO: 89 and at least 20 contiguous nucleotides from SEQ ID NO: 90 operably linked to the Arabidopsis histone H4 promoter (Ph4A748abc) in the sense orientation, (ii) a spacer sequence, e.g., intron 1 (SEQ ID NO: 94) of hpr-Gm, (iii) the polynucleotide strand of (i) in the anti-sense orientation, and (iv) a transcription terminator sequence.

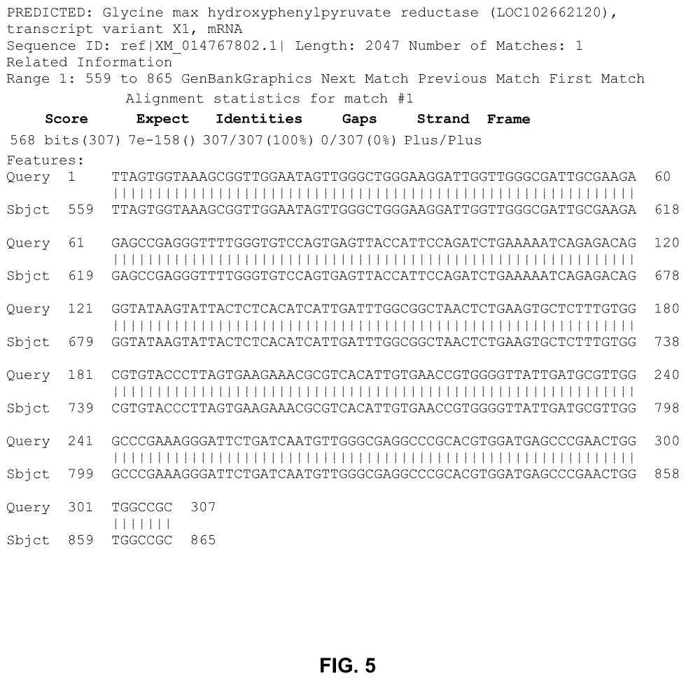

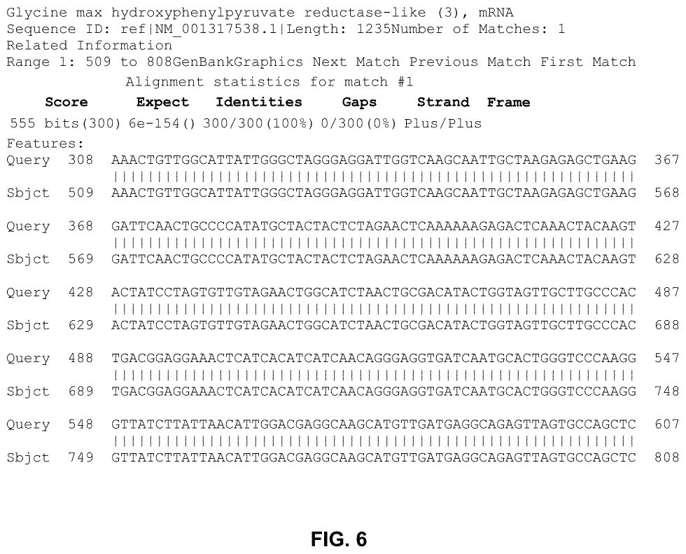

FIG. 5 shows an alignment of the concatemer sequence used in the RNAi cassette of pCPE825 (nt 1-307 of SEQ ID NO: 91) and the endogenous putative HPPR gene LOC102662120 (SEQ ID NO: 89). FIG. 6 shows an alignment of the concatemer sequence used in the RNAi cassette of pCPE825 (nt 308-607 of SEQ ID NO: 91) and the endogenous putative HPPR gene LOC100779623 (SEQ ID NO: 90). These alignments confirm that the first 307 nucleotides of the concatemer sequence of SEQ ID NO: 91 is 100% identical to nucleotides 559-865 of endogenous putative HPPR gene LOC102662120 and the last 300 nucleotides of the concatemer sequence of SEQ ID NO: 91 is 100% identical to nucleotides 509-808 of endogenous putative HPPR gene LOC100779623. RNA transcribed from the sense and antisense strands hybridize to form a hairpin structure that functions as a microRNA targeting hpr-Gm mRNA to inhibit the expression of the endogenous putative HPPR genes.

Generation of Transgenic Events and Herbicide Evaluation in T0 Lines

T0 events were generated by transforming soybean plant cells with pCPE825 (FIG. 4) using Tembotrione as selection marker. All transgenic events displayed normal phenotype.

To evaluate tolerance to HPPD inhibitor pesticides, thirty-three single copy events were sprayed with the HPPD inhibitor, NOC115. Five days following treatment, leaf damage was scored. The constructs used in this experiment are described in Table 1.

TABLE 1

| Construct | Promoter | GOI |
|---|---|---|
| pCPE825 | CsVMV/PhA748abc | Pf-evo41/HPPR-RNAi |
| pJPL0046 | CsVMV | Pf-evo41 |
| pBay00711 | CsVMV/Ph4A748abc | Pf-evo41 |

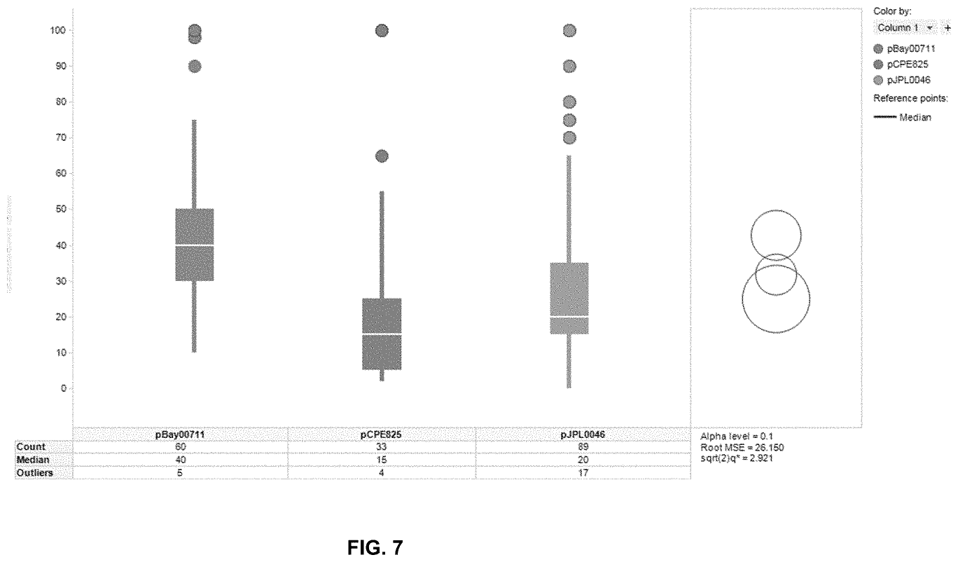

The damage ratings for the T0 events following NOC115 treatment are presented in FIG. 7. The median damage rating for the pCPE825 transformants is about 15. In contrast, in the controls, which were transformed with a construct (pBay00711) identical to pCPE825 except not containing the RNAi, the median damage rating is about 40. Thus, silencing of the putative HPPR genes resulted in a significant increase in tolerance to NOC115.

Similar results are seen in comparison to soybean plants transformed with a single Pf-HPPD-evo41 construct (pJPL0046) driven by a different promoter (CsVMV) without the RNAi cassette. For the pJPL0046 transformants, the median damage rating is about 20.

The above TO data shows a significant improvement in tolerance to the HPPD inhibitor pesticide, NOC115, by inhibiting expression of two putative HPPR genes.

These data were confirmed in a field trial of the T1 generation. Of the constructs tested in Puerto Rico, pMLS0519 (Pf-KGEPHSVV; single cassette construct encoding an improved mutant of Pseudomonas HPPD) and pCPE825 (Pf-Evo41+HPPR RNAi) were the most effective in providing good tolerance to isoxaflutole (IFT) and to NOC115.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for conferring tolerance to a 4-hydroxyphenylpyruvate dioxygenase (HPPD) inhibitor herbicide in a plant, comprising reducing expression of at least one 4-hydroxyphenylpyruvate reductase (HPPR) enzyme in the plant.

2. The method according to claim 1, wherein the method further comprises expressing a mutant HPPD enzyme, wherein the mutant HPPD enzyme is less sensitive to HPPD inhibitors than the native HPPD enzyme before mutation.

3. The method according to claim 2, wherein the mutant HPPD enzyme has the amino acid sequence of SEQ ID NO: 1 with the following amino acid substitutions:
(a) a proline at the amino acid position corresponding to amino acid position 335 of SEQ ID NO:1 and a tryptophan at the amino acid position corresponding to amino acid position 336 of SEQ ID NO:1;
(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;
(c) a tryptophan at the amino acid position corresponding to amino acid position 188 of SEQ ID NO:1 and a tryptophan at the amino acid position corresponding to amino acid position 336 of SEQ ID NO:1;
(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1; or
(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1.

4. The method according to claim 3, wherein the mutant HPPD enzyme has the amino acid sequence of SEQ ID NO: 16.

5. The method according to claim 1, wherein the expression of at least one HPPR enzyme in the plant is reduced by silencing one or more endogenous HPPR genes in the plant.

6. The method according to claim 1, wherein the plant is selected from the group consisting of: maize, sorghum, wheat, sunflower, tomato, crucifers, peppers, potato, cotton, rice, soybean, sugarbeet, sugarcane, tobacco, barley, and oilseed rape.

7. The method according to claim 1, wherein the plant is soybean.

8. The method according to claim 7, wherein the HPPR enzyme is SEQ ID NO: 92 and/or SEQ ID NO: 93.

9. The method according to claim 7, wherein the method comprises:
(a) transforming a soybean cell with a DNA construct comprising a RNAi region for inhibiting the expression of one or more endogenous HPPR genes; and
(b) regenerating a transgenic soybean plant from said transformed soybean cell.

10. The method according to claim 9, wherein the DNA construct comprising a RNAi region for inhibiting the expression of one or more endogenous HPPR genes further comprises a coding region that encodes a mutant HPPD enzyme, wherein the mutant HPPD enzyme mutant is less sensitive to HPPD inhibitors than the native HPPD enzyme before mutation.

11. The method according to claim 10, wherein the mutant HPPD enzyme has the amino acid sequence of SEQ ID NO:1 with the following amino acid substitutions:
(a) a proline at the amino acid position corresponding to amino acid position 335 of SEQ ID NO:1 and a tryptophan at the amino acid position corresponding to amino acid position 336 of SEQ ID NO:1;
(b) a serine at the amino acid position corresponding to amino acid position 335 of SEQ ID NO:1, a serine at the amino acid position corresponding to amino acid position 336 of SEQ ID NO:1, a threonine at the amino acid position corresponding to amino acid position 339 of SEQ ID NO:1, and a glutamine at the amino acid position corresponding to amino acid position 340 of SEQ ID NO:1;
(c) a tryptophan at the amino acid position corresponding to amino acid position 188 of SEQ ID NO:1 and a tryptophan at the amino acid position corresponding to amino acid position 336 of SEQ ID NO:1;
(d) a proline at the amino acid position corresponding to amino acid position 335 of SEQ ID NO:1, a serine at the amino acid position corresponding to amino acid position 336 of SEQ ID NO:1, and a glutamic acid at the amino acid position corresponding to amino acid position 340 of SEQ ID NO:1; or
(e) a proline at the amino acid position corresponding to amino acid position 335 of SEQ ID NO:1, a tryptophan at the amino acid position corresponding to amino acid position 336 of SEQ ID NO:1, an alanine at the amino acid position corresponding to amino acid position 339 of SEQ ID NO:1, and a glutamine at the amino acid position corresponding to amino acid position 340 of SEQ ID NO:1.

12. The method according to claim 10, wherein the coding region encodes the amino acid sequence set forth in any of SEQ ID NOs: 3-59 or fragments thereof.

13. The method according to claim 10, wherein the coding region encodes the amino acid sequence set forth in SEQ ID NO: 16 or fragments thereof.

14. The method according to claim 10, wherein the coding region is a synthetic sequence that has been designed for expression in soybean.

15. A transgenic soybean plant produced by the method of claim 9.
